(19) United States
(12) Patent Application Publication
(10) Pub. No.: US 2017/0239294 A1
(43) Pub. Date: Aug. 24, 2017

(54) COMPOSITIONS AND METHODS FOR TREATING B-LYMPHOID MALIGNANCIES

(71) Applicant: THE CHILDREN'S HOSPITAL OF PHILADELPHIA, Philadelphia, PA (US)

(72) Inventors: Andrei Thomas-Tikhonenko, Philadelphia, PA (US); Stephan Grupp, Havertown, PA (US); John Maris, Philadelphia, PA (US); David Barrett, Philadelphia, PA (US); Elena Sotillo-Pineiro, Ardmore, PA (US)

(73) Assignees: Novartis AG, Basel (CH); THE TRUSTEES OF THE UNIVERSITY OF PENNSYLVANIA, Philadelphia, PA (US)

(21) Appl. No.: 15/519,323

(22) PCT Filed: Oct. 15, 2015
(86) PCT No.: PCT/US15/55764

§ 371 Date: Apr. 14, 2017

Related U.S. Application Data
(60) Provisional application No. 62/064,131, filed on Oct. 15, 2014.

(51) Int. Cl. A61K 35/17; A61K 45/06; A61K 31/506

(52) U.S. Cl. CPC A61K 35/17; A61K 31/506; A61K 45/06

(57) ABSTRACT

Compositions and methods for inhibiting, treating, and/or preventing a B-cell neoplasm are provided.

This application claims priority under 35 U.S.C. §119(e) to U.S. Provisional Patent Application No. 62/064,131, filed on Oct. 15, 2014. The foregoing application is incorporated by reference herein.

### FIELD OF THE INVENTION

The present invention relates to the field of cancer therapy. Specifically, compositions and methods for inhibiting, treating, and/or preventing cancer are disclosed.

### BACKGROUND OF THE INVENTION

Several publications and patent documents are cited throughout the specification in order to describe the state of the art to which this invention pertains. Each of these citations is incorporated herein by reference as though set forth in full.

Patients with relapsed and chemotherapy-refractory pre-B-cell acute lymphoid leukemia (ALL) have a poor prognosis (Barrett et al. (1994) N. Engl. J. Med., 331:1253-1258; Gokbuget et al. (2012) Blood 120:2032-2041; Bargou et al. (2008) Science 321:974-977). However, chimeric antigen receptor-modified T cells that target CD19 (CTL019 or CART19) have shown to be an effective therapy against certain leukemias including ALL (Kochenderfer et al. (2010) Blood 116:4099-4102; Brentjens et al. (2011) Blood 118:4817-4828; Porter et al. (2011) N. Engl. J. Med., 365:725-733; Kalos et al. (2011) Sci. Transl. Med., 3:95ra73-95ra73; Grupp et al. (2013) N. Engl. J. Med., 368:1509-1518). CART19 is a chimeric antigen receptor that includes a CD137 (4-1BB) signaling domain and is expressed with the use of lentiviral-vector technology (Milone et al. (2009) Mol. Ther., 17:1453-1464). However, so-called CD19 negative relapses have been observed after CART19 treatment. Accordingly, improved methods of treating CD19 related cancers such as ALL are needed.

### SUMMARY OF THE INVENTION

In accordance with the present invention, methods of inhibiting, treating, and/or preventing cancer in a subject are provided. In a particular embodiment, the cancer is a B-cell neoplasm. In a particular embodiment, the B-cell neoplasm expresses a CD19 isoform (e.g., Δ exon2 or Δ exon 5-6), particularly without substantial or any wild-type CD19. In a particular embodiment, the method comprises administering to the subject at least one Src family kinase (SFK) inhibitor (e.g., dasatinib) and/or at least one chimeric antigen receptor-modified T cell and/or chimeric antigen receptor which recognizes the CD19 isoform, CD20, and/or CD22. In a particular embodiment, the B-cell neoplasm is a B-cell acute lymphoblastic leukemia and/or is a relapse after CART19 therapy.

In accordance with another aspect of the instant invention, diagnostic methods are provided for assessing whether a subject can be treated by the therapeutic methods of the instant invention.

### BRIEF DESCRIPTIONS OF THE DRAWING

FIGS. 1A-1D show the molecular analysis of CHOP101 and 101R samples. FIG. 1A provides primary bone marrow flow cytometry profiles gated on CD45³⁰ blasts from pre- and post- CART19 therapy, demonstrating the emergence of a CD19 negative population. FIG. 1B shows genomic DNA PCR amplifying indicated CD19 gene segments from CHOP101/101R leukemias. Prior to analysis, both samples were engrafted in NSG mice. FIG. 1C shows qRT-PCR analysis performed on cDNA from the same samples. FIG. 1D shows immunoblotting performed on the same samples. Various anti-CD19 antibodies were used in this experiment. Full-length CD19 migrates at the apparent molecular weight of 90 kDa.

FIGS. 2A-2C provide a molecular analysis of additional relapse samples. FIG. 2A shows profiling of CD19 expression by flow cytometry with the FMC63 antibody. Xenografted samples were used in this and all subsequent experiments. FIG. 2B shows qRT-PCR analysis performed on cDNA from the same samples. FIG. 2C shows immunoblotting performed on the same samples. Various anti-CD19 antibodies were used in this experiment. Full-length CD19 migrates at the apparent molecular weight of 90 kDa.

FIGS. 3A-3B provide multiple isoforms of CD19. FIG. 3A shows splice variants of CD19 mRNA reported in ENSEMBL. FIG. 3B shows a schematic of CD19. The ectodomain of CD19 recognized by the FMC antibody is shaded. The Ig-like domains are represented by loops. Exons are also depicted.

FIGS. 4A-4B show the validation of the Δex2 CD19 splice isoform. FIG. 4A shows RT-PCR on cDNA from primary and relapsed leukemias using primers in exons 1 and 3. Xenografted samples were used in this experiment. FIG. 4B shows RNASeq analysis of human samples, showing alignment of reads to CD19 exons. Circled is the number of reads that skip exon 2.

FIGS. 5A-5B show the validation of the Δex5-6 CD19 splice isoform. FIG. 5A shows RT-PCR on cDNA from primary and relapsed leukemias using primers in Exons 4 and 7. Xenografted samples were used in this experiment. FIG. 5B shows RNASeq analysis of human samples, showing alignment of reads to CD19 exons. Circled is the number of reads that skip exons 5 and 6.

FIGS. 6A-6B shows the loss of CD19 FMC63 epitope and implications for therapy. FIG. 6A shows the summary of patient samples analyzed. Shown in columns are CD19 expression patterns at the protein and mRNA levels, with emphasis on alternative splicing events. FIG. 6B shows the detection of CD19 surface expression by flow cytometry in patient sample CHOP107R. Two different antibodies were used in this experiment.

FIG. 7A: Flow cytometric profiles of CD19 surface expression in paired BALL samples included in subsequent analyses. FIG. 7B: CD19 gene coverage obtained through whole genome sequencing of CHOP101 and CHOP101R samples. FIG. 7C: SNP array analysis of Chr16p performed on DNA from 105R1 and 105R2 showing the large hemizygous deletion (brackets) found in the CHOP105R2 sample. FIG. 7D: Direct bisulfite sequencing of the enhancer and promoter regions of CD19 (downstream of the Pax5 binding site) in the paired samples. A CpG island within the HOXA3 locus was analyzed as a positive control. FIG. 7E: qRT-PCR analysis of Pax5 mRNA expression in xenografted patient samples. Actin and GAPDH were used as reference genes. FIG. 7F: qRT-PCR analysis of different regions of the CD19 mature mRNA. In all qPCR panels, graphs show relative quantifications of expression ±1 S.D. FIG. 7G: Genome browser SIB track predicted isoforms of CD19 mRNA, including those skipping exon 2 (Δex2) and exons 5 and 6 (Δex5-6), and the partial deletion of exon 2 (ex2part) that shifts the reading frame.

FIG. 8A: Levels of CD19 mRNA in xenografts of paired pre- and post-CART-19 B-ALL samples. Values represent reads per kilobase per million mapped reads (rpkm). FIG. 8B, top: Splicegraphs of CD19 mRNA species from primary (CHOP101) and relapsed (CHOP101R) tumors. Shown above arcs are raw numbers of RNA-Seq reads spanning annotated and novel splice junctions. Bottom: Violin plots showing the distribution of PSI values (Y-axis) quantified by MAJIQ for primary (101, left) and relapsed (101R, right) samples. Shades correspond to the junctions displayed in the thumbnail (far left) with the expected PSI value for each junction displayed on the X-axis. FIG. 8C: Analysis by low-cycle semi-quantitative RT-PCR of the region spanning exons 4 to 8. cDNA were obtained from paired primary and relapsed samples. CD19-negative JSL1 T-cell line was used as negative control. Arrows indicate inclusion of exons 5-6 (+) and the Δex5-6 isoform. FIG. 8D: Semi-quantitative RT-PCR of cDNA from xenografted samples corresponding to exons 1-4 of CD19. Arrows indicate full length (FL), partial deletion (ex2part) and the Δex2 isoform. Quantification of relative isoform abundance in each sample (numbers below) was performed using Image J software (NIH). FIG. 8E: qRT-PCR analysis of CD19 splicing variants using oligos that span conserved and alternative exon/exon junctions. Graph shows relative quantification of expression±1 S.D. Oligos expanding exon3/4 of CD19 were used as reference. FIG. 8F: Semi-quantitative RT-PCR of cDNA from xenografted samples corresponding to exons 1-5 of CD19. FIG. 8G: Direct Sanger sequencing performed from gel-purified bands in FIG. 8F. Exon1/3 junction (left) and single nucleotide insertion in exon2 (right) are indicated. FIG. 8H: qRT-PCR analysis of CD19 splicing variants using oligos as in FIG. 8E, in cDNA from 697 cells were CD19 exon2 was targeted and mutated using CRISPR/Cas9.

FIG. 9A: Detail from Sanger sequencing of exon4-8 cDNA obtained from xenografted samples showing enhanced skipping of exons 5 and 6 in the relapse CHOP101R sample. FIG. 9B: Detail of Sanger sequencing of the exon 1-4 cDNA showing Exon^{2/}₃junction. Major traces align with exon2-exon3 in CHOP101 (top), while exon 1-exon3 junction dominates in sample CHOP101R (bottom). FIG. 9C: Detail of Sanger sequencing of the exon1-4 cDNA from sample CHOP133R showing that only exon1-3 junction is detectable. This sample has a hemizygous deletion of Chromosome 16 and the remaining CD19 allele carries a nonsense mutation in exon 2. FIG. 9D: Summary of mutations found in post-CART19 relapsed leukemias along CD19 exon 2. Highlighted is the CRISPR/Cas9 targeted sequence used to introduce mutations in exon2. FIG. 9E: Immunoblotting for CD19 in protein lysates from a panel of human lymphoid B cell lines that were targeted with CRISPR/Cas9-CD19exon2-gRNA. Arrows indicate full length (FL) and the Δex2 isoform. The antibody used (Cell signaling) recognizes the cytosolic domain. FIG. 9F: qRT-PCR analysis of CD19 splicing variants in Nalm-6 (left) and Raji (right) cells targeted with CRISPR/Cas9 as in FIG. 9E. Oligos used span conserved and alternative exon/exon junctions. Graph shows relative quantification of expression±1 .S.D. Oligos expanding exon3/4 of CD19 were used as reference.

FIG. 10A: Splicing factors predicted by the AVISPA algorithm to process introns 1 and 2 and introns 4, 5, and 6 of the CD19 mRNA. Numbers represent the predicted normalized feature effect (NFE) score, shades represent contribution of the binding motifs in the matching regions. Highlighted are those factors that overlap in all three analyzed cassettes. FIG. 10B: RNA pull-down assay for detection of splicing factors present in nuclear extracts of B cells that bind to CD19-exon2 and its flanking introns. Input lane shows pattern of bands corresponding to all nuclear proteins that bind the CD19-minigene. Putative splicing factors with molecular sizes similar to the bands detected are listed. (*) Indicates those that were also predicted by AVISPA. FIG. 10C: RNA-immunoprecipitations were performed using antibodies against indicated proteins. Numbers in parentheses indicated expected molecular weights for each protein. FIG. 10D: Efficiency of siRNA knock-down measured by qRT-PCR in RNA from P493-6 cells transfected with increasing concentrations of indicated siRNAs. FIG. 10E: qRT-PCR analysis of CD19Dex2 splicing variant in RNA from P493-6 transfected with increasing concentrations of si-SRSF3 or si-hRNPC.

FIG. 11A: Venn diagrams of splicing factors predicted by CD19mRNA pull-down (biochemical predictions) or by the sequence-based algorithm AVISPA to bind to CD19 exon1-exon3 (splicing of exon2) or exon4-exon7 (splicing of exons 5-6) mRNA of CD19. FIG. 11B: RNA-immunoprecipitation with antibodies against indicated proteins for detection of splicing factors that bind to mRNA CD19-exon2 and its flanking introns. Numbers in parentheses indicated expected molecular weights for each protein. FIG. 11C: Increasing concentrations of siRNAs targeting SRSF3 were transfected into Nalm-6 cells. Efficiency of siRNA knock-down was measured by qRT-PCR 24 hours after transfection. FIG. 11D: RNAs from samples treated as in FIG. 11C, were extracted and the amount of CD19 Δex2 splicing was measured by qRT-PCR. FIG. 11E: Immunoblotting for CD19 and SRSF3 in protein lysates from indicated cell lines transfected with increasing concentrations of si-SRSF3 for 24 hours. Arrows indicate full length (FL) and exon 2-skipping (Δex2) CD19 variants. Quantification of SRSF3 and Δex2 abundance relative to siRNA controls is shown. FIG. 11F: Violin plots showing the distribution of PSI values (Y-axis) quantified by MAJIQ for control (left) and SRSF-3 knockdown (right) GM19238 B-cells. Shades correspond to the junctions displayed in the thumbnail (far left) with the expected PSI value for each junction displayed on the X-axis. FIG. 11G: Immunoblotting of SRSF3 in xenografted tumor samples. Quantification of relative SRSF3 protein abundance (numbers on top) was performed using Image J software (NIH).

FIG. 12A: CD19 proteins encoded by the full length and the Δex2 and Δex5-6 isoforms of CD19 mRNA. The epitope recognized by CART-19 is encoded by exons 1 and 2. The transmembrane domain is encoded by exons 5 and 6. FIG. 12B: Immunoblotting for CD19 in protein lysates from xenografted tumor samples using antibodies recognizing the extracellular domain (clone 3F5 from Origene) [top panel] or the cytosolic domain (Santa Cruz Biotechnologies sc-69735) [bottom panel]. FIG. 12C: Immunoblotting for CD19 in protein lysates from a panel of cell lines representing human B cell malignancies. Arrows indicate full length (FL) and the Δex2 isoform. The antibody used (Santa Cruz Biotechnologies sc-69735) recognizes the cytosolic domains. FIG. 12D: Retroviral constructs generated to ectopically express full-length and truncated isoforms of CD19, with or without GFP. FIG. 12E: Immunoblotting for CD19 in lysates from CD19-negative Myc5 murine B lymphoid cells transduced with CD19 retroviral constructs. Arrows indicate full length (FL), Δex2 and Δex5-6 isoforms. FIG. 12F: Flow cytometry performed on CD19-negative murine Myc5 cells infected with empty, full length CD19, or CD19 Δex2 expressing retrovirus. FIG. 12G: Growth rates of first three cultures from FIG. 12D. Average fold increase in cell numbers from triplicate plates is shown. Statistical significance per Student's t-test, with *p≦0.05 and **p<0.01. FIG. 12H: qRT-PCR analysis of CD19 splicing variants in Nalm-6 that were treated with Actinomycin D for indicated periods of time. Myc mRNA was used as internal control for effective inhibition of transcription. FIG. 12I: Immunoblotting analysis of CD19 protein stability in cells from FIG. 12E. Cultures were treated with cycloheximide for indicated periods of time. Labile Myc protein was used as control for effective inhibition of protein synthesis.

FIG. 13A: Flow cytometry analysis of CD19 expression on the surface of parental and CD19-negative Nalm-6 cells. FIG. 13B: Immunoblotting for CD19 in lysates from CD19-negative Nalm-6 cells transduced with retroviral constructs from FIG. 12D. FIG. 13C: Immunoblotting of CD19 in protein lysates from CD19-negative 697 cells with reconstituted expression of full length of CD19 Δex2. FIG. 13D: Confocal microscopy of 697 ΔCD19 cells expressing CD19-GFP and CD19 Δex2-GFP fusion proteins. Plasma membranes and DNA were stained for co-localization studies. Histograms represent the intensity of the CD19-GFP and membrane along the cell-to-cell junction highlighted in the “merge” picture. FIG. 13E: Immunoblotting detection of the shift in CD19 protein size in lysates from CD19-negative 697 cells transduced with full length of Δex2 retroviral constructs and treated with a mix of glycosylases. FIG. 13F: Immunoblotting for CD19 in protein lysates from Nalm-6 ΔCD19 cells with reconstituted expression of full length, Δex2 or Δex5-6 CD19 variants that were incubated with trypsin. “<R” indicates bands that correspond to CD19 resistant to trypsin (intracellular), “<CLV” indicates CD19 cleaved by trypsin (plasma membrane). Quantification of CD19 resistant or sensitive to trypsin is shown. FIG. 13G: Nalm-6 ΔCD19-Luciferase+cells where infected with CD19 retroviral constructs, then incubated with CART-19 cells at indicated ratios of Effector T cells (E) to Target Nalm-6 cells (T), and cell death was assayed by measurement of Luminescence. Erythroleukemic K562 cells were used as a negative control. FIG. 13H: Immunoblotting in lysates from 697 ΔCD19 cells transduced with CD19-retroviral constructs expressing Full length (FL) or CD19 Δex2, and incubated with a-IgM or Isotype control (Iso) for indicated times. Activation of BCR-downstream signal was assessed by immunoblotting with P-AKT and total (Pan) AKT. Numbers indicate quantification of P-AKT bands relative to total AKT as measured by Odyssey Infrared Imager (LI-COR Biosciences). FIG. 13I: Growth rates of Nalm-6 ΔCD19 with reconstituted expression of CD19 as in FIG. 13D. Average fold increase in cell numbers from triplicate plates is shown. Statistical significance per Student's t-test, with *p≦0.05 and **p<0.01. FIG. 13J: Immunoblotting of CD19 present in complexes with PI3K or Lyn. These complexes were first coimmunoprecipitated from Nalm-6 ΔCD19 cells transduced with the indicated CD19 retroviral constructs. Prior to the experiment, cells were stimulated with α-IgM or control IgG for 10 minutes. FIG. 13K: Growth rates of Nalm-6 ΔCD19 with reconstituted expression of CD19 as in FIG. 13D. Average fold increase in cell numbers from triplicate plates is shown. Statistical significance per Student t test, with *, P≦0.05 and **, P<0.01. I, Nalm-6 ΔCD19-Luciferase+ cells were infected with CD19 retroviral constructs, then incubated with CART-19 cells at indicated ratios of effector T cells (E) to target Nalm-6 cells (T), and cell death was assayed by measurement of luminescence. Erythroleukemic K562 cells were used as a negative control.

FIG. 14A provides an example of a nucleotide sequence for CD19 where exons are indicated by alternating italics and underlining. FIG. 14B provides an example of an amino acid sequence of CD19 wherein the regions encoded by the exons of CD19 are indicating by alternating italics and underlining.

### DETAILED DESCRIPTION OF THE INVENTION

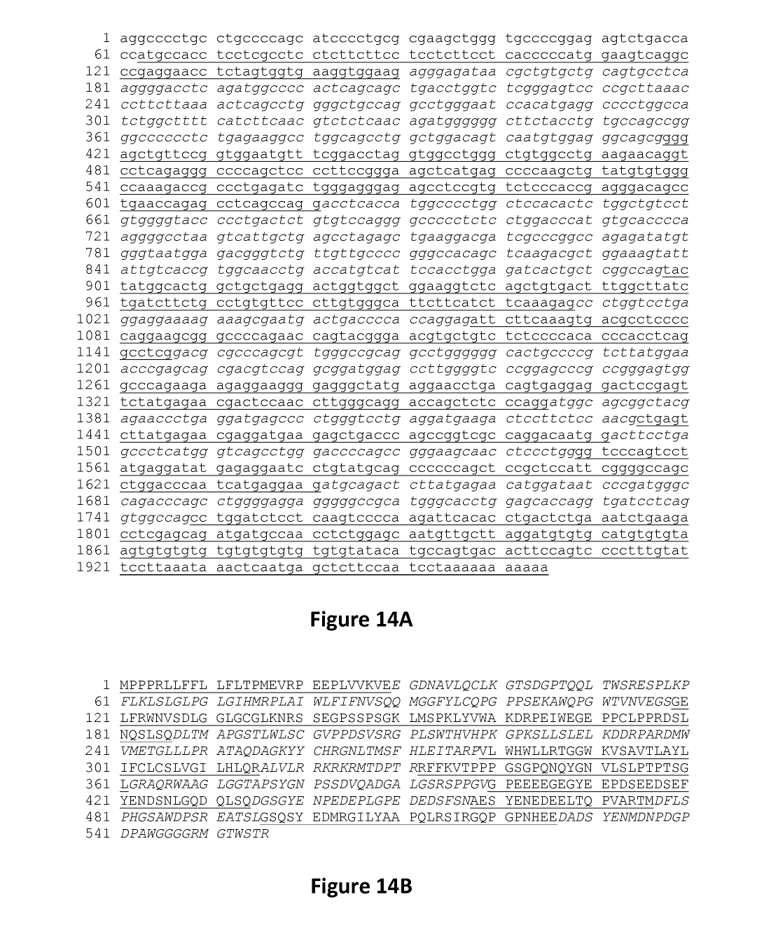

CD19 (Cluster of Differentiation 19; Gene ID: 930; UniProtKB/Swiss-Prot: P15391.6; GenBank Accession No. P15391; examples of nucleotide and amino acid sequences are provided in FIG. 14) is a well-known B cell surface molecule, which upon BCR activation enhances B-cell antigen receptor-induced signaling crucial of the expansion of B-cell population (Tedder,T. F. (2009) Nat. Rev. Rheumatol., 5:572-577). CD19 is broadly expressed in both normal and neoplastic B-cells. Because B-cell neoplasms frequently maintain CD19 expression, it (along with CD20) is regarded as the target of choice for a variety of immunotherapeutic agents, including immunotoxins (Scheuermann et al. (1995) Leuk. Lymphoma 18:385-397; Tedder, T. F. (2009) Nat. Rev. Rheumatol., 5:572-577). In particular, humanized anti-CD19 mAbs and allogeneic T-cells expressing chimeric antibody receptor for CD19 have entered clinical trials. They are presumed to work by recognizing and depleting CD19-expressing neoplastic B-cells (Davies et al. (2010) Cancer Res., 70:3915-3924; Awan et al. (2010) Blood 115:1204-1213). Notably, treatment with anti-CD19 antibodies typically results in internalization of CD19 and by inference—loss of its function (Sapra et al. (2004) Clin. Cancer Res., 10:2530-2537). Despite the promise of CART19 (CTL019) therapy, so-called CD19 negative relapses have been observed. However, as explained herein, the so-called CD19 negative relapses actually express other isoforms of CD19 which are not recognized by CART19. There CD19 isoforms can be targeted as a new means for treating relapses after CART19 therapy.

In accordance with the instant invention, methods of inhibiting (e.g., reducing), preventing, and/or treating cancer are provided. In a particular embodiment, the cancer is CD19 positive (e.g., expresses an isoform of CD19, particularly to the general exclusion of wild-type CD19 (e.g., without substantial CD19 expression)). In a particular embodiment, the cancer is CD-19 positive multiple myeloma. In a particular embodiment, the cancer is a B-cell neoplasm. B-cell neoplasms include, without limitation, lymphoma, non-Hodgkin's lymphoma, acute lymphoblastic leukemia (e.g., pre-B-cell acute lymphoblastic leukemia, B-cell acute lymphoblastic leukemia), and chronic lymphocytic leukemia. In a particular embodiment, the cancer expresses an isoform of CD19 (e.g., Δ exon2, Δ exon 5-6), particularly without substantial or any wild-type CD19 (e.g., below detection limits (e.g., by Western) or at levels insufficient to be treated by CART19). In a particular embodiment, the cancer expresses a Δ exon2 isoform of CD19. In a particular embodiment, the cancer is a relapse after CART19 treatment.

In a particular embodiment, the method of inhibiting (e.g., reducing), preventing, and/or treating cancer comprises administering a Src family tyrosine kinase inhibitor, particularly a Lyn inhibitor to a subject in need thereof. Examples of Lyn inhibitor include, without limitation, dasatinib, PP2, Lyn inhibitory nucleic acid molecules (e.g., antisense, siRNA, shRNA, etc.). The methods may further comprise administering chimeric antigen receptor-modified T cells with specificity for a CD19 isoform, CD20, and/or CD22, as described hereinbelow.

In a particular embodiment, the method of inhibiting (e.g., reducing), preventing, and/or treating cancer comprises administering chimeric antigen receptor-modified T cells with specificity for a CD19 isoform (e.g., the CD19 isoform identified in the cancer of the subject), CD22 (e.g., Haso et al. (2013) Blood 121(7):1165-74), and/or CD20. In a particular embodiment, the method of inhibiting (e.g., reducing), preventing, and/or treating cancer comprises administering chimeric antigen receptor-modified T cells with specificity for a CD19 isoform (e.g., the CD19 isoform identified in the cancer of the subject), particularly the Δ exon2 isoform of CD19. Chimeric antigen receptors typically comprise at least the antigen recognition domain of an antibody, a transmembrane domain, and an intracellular domain (e.g., a T-cell activation domain). While chimeric antigen receptor-modified T cells will generally be administered in accordance with the methods provided herein, the methods of the instant invention also comprise administering a nucleic acid (DNA or RNA) encoding a chimeric antigen receptor with specificity for a CD19 isoform, CD22, and/or CD20 to the subject. When chimeric antigen receptor-modified T cells are administered, T cells (e.g., T cell, cytotoxic T cell, and/or natural killer) comprising nucleic acid encoding a chimeric antigen receptor with specificity for a CD19 isoform, CD22, and/or CD20 are administered to the subject. The administered T cells may be autologous. For example, the methods may comprise transducing T cells ex vivo with a nucleic acid encoding a chimeric antigen receptor of the instant invention (e.g., an integrating or non-integrating vector for the expression of the chimeric antigen receptor). The methods of the instant invention may further comprise obtaining the T cells from the subject to be treated. In a particular embodiment, the method comprises the administration of an anti-CD20 antibody (e.g., rituximab). In a particular embodiment, the method comprises the administration of at least one PI3K inhibitor (e.g., wortmannin, PX-866, LY294002). In a particular embodiment, the method comprises the administration of an anti-CD19 antibody which recognized the CD19 isoform.

The methods of the instant invention may further comprise administering an agent which assists protein folding and/or prevents degradation of misfolded proteins (e.g., misfolded membrane proteins). In a particular embodiment, the agent is an activator of the unfolded protein response (UPR). Examples of such agents are described in Hetz et al. (Nature Reviews Drug Discovery (2013) 12:703-719) and include, without limitation, sunitinib, sorafenib, STF-083010, 40C, MKC-3946, toyocamycin, GSK2656157, bortezomib, MG-132, eeyarstatin, ML240, DBeQ, 17-AAG, radicicol, and MAL3-101. In a particular embodiment, the agent is administered to a subject whose cancer expresses the Δ exon2 isoform of CD19 and/or is being treated with a chimeric antigen receptor with specificity for the Δ exon2 isoform of CD19 isoform and/or T cells comprising the nucleic acid encoding a chimeric antigen receptor with specificity for the Δ exon2 isoform of CD19.

The nucleic acid encoding a chimeric antigen receptor with specificity for a CD19 isoform, CD22, and/or CD20 and/or T cells comprising the nucleic acid encoding a chimeric antigen receptor with specificity for a CD19 isoform, CD22, and/or CD20 may be administered to a subject consecutively (e.g., before and/or after) and/or simultaneously with another therapy for treating, inhibiting, and/or preventing the cancer in said subject. The additional therapy may be the administration of a chemotherapeutic agent and/or any one or more of the additional therapies described hereinabove. Kits comprising at least one first composition comprising at least one nucleic acid encoding a chimeric antigen receptor with specificity for a CD19 isoform, CD22, and/or CD20 and/or T cells comprising the nucleic acid encoding a chimeric antigen receptor with specificity for a CD19 isoform, CD22, and/or CD20 of the instant invention and at least one second composition comprising at least one other therapeutic agent are also encompassed by the instant invention.

Typically, chimeric antigen receptor-modified T cells express a single chain Fv region of a monoclonal antibody to recognize a cell-surface antigen independent of the major histocompatibility complex (MHC) coupled with one or more signaling molecules to activate genetically modified T cells for killing, proliferation, and cytokine production. Clinical trials with CAR-modified T cells for treating B cell malignancies have been reported (Porter et al. (2011) N. Engl. J. Med., 365:725-33; Grupp et al. (2013) N. Engl. J. Med., 368:1509-18). Generally, the chimeric antigen receptor comprises an ectodomain (extracellular domain), a transmembrane domain, and an endodomain (cytoplasmic or intracellular domain). The ectodomain of the chimeric antigen receptor typically comprises an antibody or fragment thereof. In a particular embodiment, the antibody or fragment thereof of the instant invention is immunologically specific for a CD19 isoform (e.g., Δ exon2 or Δ exon 5-6), CD22, and/or CD20. In a particular embodiment, the antibody or fragment thereof is immunologically specific for the extracellular domain of the target molecule, particularly the CD19 isoform. In a particular embodiment, the antibody or fragment thereof is immunologically specific for the portion of CD19 encoded by exons 1, 3, and/or 4 (see, e.g., FIG. 14). In a particular embodiment, the antibody or fragment thereof is immunologically specific for an epitope which bridges the portion of CD19 encoded by exon 1 and the portion of CD19 encoded by exon 3 (i.e., spans the region where exon 1 and exon 3 are fused). In a particular embodiment, the antibody or fragment thereof comprises a Fab or a scFv, particularly scFv. Typically, the antibody or an antigen-binding fragment of the ectodomain may be linked to the transmembrane domain via an amino acid linker/spacer (e.g., about 1 to about 100 amino acids). The ectodomain may also comprise a signal peptide (e.g., an endoplasmic reticulum signal peptide).

The transmembrane domain of the chimeric antigen receptor may be any transmembrane domain. Generally, the transmembrane domain is a hydrophobic alpha helix that spans the cell membrane and is often from the same protein as the endodomain. Examples of transmembrane domains include, without limitation, transmembrane domains from T-cell receptor (TCR), CD28, CD3, CD45, CD4, CD5, CD8, CD9, CD16, CD22, CD33, CD37, CD64, CD80, CD86, CD134, CD137, or CD154. In a particular embodiment, the transmembrane domain is from CD3-ζ or CD28.

The endodomain of a chimeric antigen receptor comprises at least one signaling domain (e.g., a signaling domain comprising one or more immunoreceptor tyrosine-based activation motifs (ITAMs)). The signaling domain is activated by antigen binding to the ectodomain and leads to the activation of the T cells. Signaling domains include, without limitation, the signaling domain (e.g., endodomain/cytoplasmic domain or fragment thereof) from CD3 (e.g., CD3-c, CD3-γ, or CD3-ζ), LIGHT, lymphocyte function-associated antigen 1 (LFA-1), CD2, CD28, ICOS, CD30, CD7, NKG2C, CD40, PD-1, OX40, CD18, CD27, B7-H3, 4-1BB, OX40, CD40, and NKG2C. In a particular embodiment, the endodomain comprises more than one signaling domain. In a particular embodiment, the endodomain comprises the signaling domains of CD3-ζ, CD28, 4-1BB, and/or OX40. In a particular embodiment, the endodomain comprises the signaling domains of CD3-ζ, CD28, and 4-1BB.

Nucleic acid molecules encoding the chimeric antigen receptor of the instant invention may be contained within a vector (e.g., operably linked to a promoter and/or enhancer for expression in the desired cell type). The vector may be DNA or RNA. The vector may be an integrating vector or a non-integrating vector. Examples of vectors include, without limitation, plasmids, phagemids, cosmids, and viral vectors. In a particular embodiment, the vector is a viral vector. Examples of viral vectors include, without limitation: a parvoviral vector, lentiviral vector (e.g., HIV, SIV, FIV, EIAV, Visna), adenoviral vector, adeno-associated viral vector (e.g., AAV1-9), herpes vector (HSV1-8), or a retroviral vector. The viral vector may be a psuedotype viral vector. For example, the vector may be a SIV or HIV based, VSVG pseudo-typed lentiviral vector. The promoter of the vector may be constitutive or inducible. Examples of promoters include, without limitation: the immediate early cytomegalovirus (CMV) promoter, elongation growth factor-1α, simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, actin promoter, myosin promoter, hemoglobin promoter, creatine kinase promoter, metallothionine promoter, glucocorticoid promoter, progesterone promoter, and tetracycline promoter.

For ex vivo methods, the nucleic acid molecules (e.g., vectors) of the instant invention may be transferred into the desired target cell (e.g., T cell) by any physical, chemical, or biological means. Methods for transferring nucleic acid molecules into cells are well known in the art (see, e.g., Sambrook et al. (Molecular Cloning: A Laboratory Manual, Cold Spring Harbor Laboratory, New York). Exemplary methods of transferring the nucleic acid molecules into cells include, without limitation: calcium phosphate precipitation, lipofection, particle bombardment, microinjection, electroporation, infection (e.g., with viral vector), and colloidal dispersion systems (e.g., nanocapsules, microspheres, micelles, and liposomes).

The methods of the instant invention may also comprise determining the CD19 (e.g., wild-type and/or isoform) expressed by the cancer prior to treatment of the subject. The method may further comprise obtaining a biological sample (e.g., blood) from said subject. The CD19 isoform expressed can be determined by any method known in the art including, without limitation, sequencing (e.g., all or part (e.g., ectodomain) of CD19), isoform specific PCR, isoform-specific oligonucleotide or probe screening methods, recognition by isoform specific antibodies, etc.

As stated hereinabove, the methods of the instant invention may further comprise the administration of at least one other cancer therapy (simultaneously and/or sequentially (before and/or after)) such as radiation therapy and/or the administration of at least one other chemotherapeutic agent. Chemotherapeutic agents are compounds that exhibit anticancer activity and/or are detrimental to a cell (e.g., a toxin). Suitable chemotherapeutic agents include, but are not limited to: toxins (e.g., saporin, ricin, abrin, ethidium bromide, diptheria toxin, Pseudomonas exotoxin, and others listed above; thereby generating an immunotoxin when conjugated or fused to an antibody); monoclonal antibody drugs (e.g., rituximab, cetuximab); alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide).

The compositions of the present invention can be administered by any suitable route, for example, by injection (e.g., for local (direct, including to or within a tumor) or systemic administration), oral, pulmonary, topical, nasal or other modes of administration. The composition may be administered by any suitable means, including parenteral, intramuscular, intravenous, intraarterial, intraperitoneal, subcutaneous, topical, inhalatory, transdermal, intrapulmonary, intraareterial, intrarectal, intramuscular, and intranasal administration. In a particular embodiment, the composition is administered to the blood (e.g., intravenously). In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., Tween® 80, polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (e.g., Remington: The Science and Practice of Pharmacy). The pharmaceutical composition of the present invention can be prepared, for example, in liquid form, or can be in dried powder form (e.g., lyophilized for later reconstitution).

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the molecules to be administered, its use in the pharmaceutical preparation is contemplated.

The dose and dosage regimen of the molecule of the invention that is suitable for administration to a particular patient may be determined by a physician considering the patient's age, sex, weight, general medical condition, and the specific condition and severity thereof for which the inhibitor is being administered. The physician may also consider the route of administration, the pharmaceutical carrier, and the molecule's biological activity.

Selection of a suitable pharmaceutical preparation depends upon the method of administration chosen. For example, the molecules of the invention may be administered by direct injection into any cancerous tissue or into the area surrounding the cancer. In this instance, a pharmaceutical preparation comprises the molecules dispersed in a medium that is compatible with the cancerous tissue.

Molecules of the instant invention may also be administered parenterally by intravenous injection into the blood stream, or by subcutaneous, intramuscular, intrathecal, or intraperitoneal injection. Pharmaceutical preparations for parenteral injection are known in the art. If parenteral injection is selected as a method for administering the molecules, steps should be taken to ensure that sufficient amounts of the molecules reach their target cells to exert a biological effect. The lipophilicity of the molecules, or the pharmaceutical preparation in which they are delivered, may have to be increased so that the molecules can arrive at their target locations. Methods for increasing the lipophilicity of a molecule are known in the art.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral, topical, or parenteral. In preparing the molecule in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric-coated by standard techniques. For parenterals, the carrier will usually comprise sterile water, though other ingredients, for example, to aid solubility or for preservative purposes, may be included. Injectable suspensions may also be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Various concentrations of pharmaceutical preparations may be administered to mice with transplanted human tumors, and the minimal and maximal dosages may be determined based on the results of significant reduction of tumor size and side effects as a result of the treatment. Appropriate dosage unit may also be determined by assessing the efficacy of the treatment in combination with other standard chemotherapies. The dosage units of the molecules may be determined individually or in combination with each chemotherapy according to greater shrinkage and/or reduced growth rate of tumors.

The pharmaceutical preparation comprising the molecules of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

In accordance with another aspect of the instant invention, methods of identifying agents which target CD19 isoforms (e.g., CD19 isoforms identified after CART19 relapse and/or those CD19 isoforms described herein) are provided. In a particular embodiment, the screening methods of the instant invention comprise performing a binding assay in the presence of the CD19 isoform (including cells expressing the CD19 isoform (e.g., isolated from a subject after CART19 relapse)) to identify compounds which can specifically bind the CD19 isoform. Binding assays include, without limitation, cell surface receptor binding assays, fluorescence energy transfer assays, liquid chromatography, membrane filtration assays, ligand binding assay, radiobinding assay, immunoprecipitations, radioimmunoassays, enzyme-linked immunosorbent assays (ELISA), immunohistochemical assays, Western blot, and surface plasmon resonance. In a particular embodiment, the CD19 isoform is immobilized (e.g., to a solid support) in the binding assay. In a particular embodiment, the test agent is an antibody, small molecule or a peptide, particularly an antibody.

In accordance with another aspect of the instant invention, methods of diagnosing a cancer are also provided. The methods can be used to determine whether a subject should be treated with wild-type CART19 therapy or a therapeutic method of the instant invention. In a particular embodiment, the method comprises determining whether the cancer (e.g., a B cell) expresses wild-type CD19 and/or a CD19 isoform, wherein the presence of a CD19 isoform and/or absence of wild-type CD19 indicates that the cancer will be refractory to CART19 (wild-type) therapy. Methods of determining whether a B cell expresses wild-type CD19 or a CD19 isoform are described herein and include, without limitation, sequencing (e.g., all or part (e.g., ectodomain) of CD19), isoform specific PCR, isoform-specific oligonucleotide or probe screening methods, recognition by isoform specific antibodies, etc. The method may further comprise treating the subject in accordance with the therapeutic methods of the instant invention.

### Definitions

The following definitions are provided to facilitate an understanding of the present invention:

The singular forms “a,” “an,” and “the” include plural referents unless the context clearly dictates otherwise.

As used herein, the terms “host,” “subject,” and “patient” refer to any animal, particularly mammals including humans.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween® 80, polysorbate 80), emulsifier, buffer (e.g., Tris HCl, acetate, phosphate), antimicrobial, bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in, for example, Remington: The Science and Practice of Pharmacy; Liberman, et al., Eds., Pharmaceutical Dosage Forms; and Rowe, et al., Eds., Handbook of Pharmaceutical Excipients.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., cancer) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof.

As used herein, the term “subject” refers to an animal, particularly a mammal, particularly a human.

As used herein, the term “small molecule” refers to a substance or compound that has a relatively low molecular weight (e.g., less than 4,000, particularly less than 2,000). Typically, small molecules are organic, but are not proteins, polypeptides, or nucleic acids, though they may be amino acids or dipeptides.

An “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule. The term includes polyclonal, monoclonal, chimeric, single domain (Dab) and bispecific antibodies. As used herein, antibody or antibody molecule contemplates recombinantly generated intact immunoglobulin molecules and molecules comprising immunologically active portions of an immunoglobulin molecule such as, without limitation: Fab, Fab′, F(ab′)₂, F(v), scFv, scFv₂, scFv-Fc, minibody, diabody, tetrabody, and single variable domain (e.g., variable heavy domain, variable light domain). Methods of making antibodies directed toward a target polypeptide or protein or fragment thereof (e.g., epitope) are well known in the art.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

The phrase “solid support” refers to any solid surface including, without limitation, any chip (for example, silica-based, glass, or gold chip), glass slide, membrane, plate, bead, solid particle (for example, agarose, sepharose, polystyrene or magnetic bead), column (or column material), test tube, or microtiter dish.

The term “vector” refers to a carrier nucleic acid molecule (e.g., DNA) into which a nucleic acid sequence can be inserted for introduction into a host cell where it will be replicated. The vector may contain a gene or nucleic acid sequence with the necessary regulatory regions needed for expression in a host cell.

The term “operably linked” means that the regulatory sequences necessary for expression of a coding sequence are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

The term “substantially pure” refers to a preparation comprising at least 50-60% by weight the compound of interest (e.g., nucleic acid, oligonucleotide, protein, etc.), particularly at least 75% by weight, or at least 90-99% or more by weight of the compound of interest. Purity may be measured by methods appropriate for the compound of interest (e.g. chromatographic methods, agarose or polyacrylamide gel electrophoresis, HPLC analysis, and the like).

As used herein, a “linker” is a chemical moiety comprising a covalent bond or a chain of atoms that covalently attaches two molecules to each other. In a particular embodiment, the linker comprises amino acids, particularly from 1 to about 25, 1 to about 20, 1 to about 15, or 1 to about 10 amino acids.

The phrase “small, interfering RNA (siRNA)” refers to a short (typically less than 30 nucleotides long, particularly 12-30 or 20-25 nucleotides in length) double stranded RNA molecule. Typically, the siRNA modulates the expression of a gene to which the siRNA is targeted. Methods of identifying and synthesizing siRNA molecules are known in the art (see, e.g., Ausubel et al. (2006) Current Protocols in Molecular Biology, John Wiley and Sons, Inc). As used herein, the term siRNA may include short hairpin RNA molecules (shRNA). Typically, shRNA molecules consist of short complementary sequences separated by a small loop sequence wherein one of the sequences is complimentary to the gene target. shRNA molecules are typically processed into an siRNA within the cell by endonucleases. Exemplary modifications to siRNA molecules are provided in U.S. Application Publication No. 20050032733. Expression vectors for the expression of siRNA molecules preferably employ a strong promoter which may be constitutive or regulated. Such promoters are well known in the art and include, but are not limited to, RNA polymerase II promoters, the T7 RNA polymerase promoter, and the RNA polymerase III promoters U6 and H1 (see, e.g., Myslinski et al. (2001) Nucl. Acids Res., 29:2502 09).

“Antisense nucleic acid molecules” or “antisense oligonucleotides” include nucleic acid molecules (e.g., single stranded molecules) which are targeted (complementary) to a chosen sequence (e.g., to translation initiation sites and/or splice sites) to inhibit the expression of a protein of interest. Such antisense molecules are typically between about 15 and about 50 nucleotides in length, more particularly between about 15 and about 30 nucleotides, and often span the translational start site of mRNA molecules. Antisense constructs may also be generated which contain the entire sequence of the target nucleic acid molecule in reverse orientation. Antisense oligonucleotides targeted to any known nucleotide sequence can be prepared by oligonucleotide synthesis according to standard methods.

The following examples provide illustrative methods of practicing the instant invention, and are not intended to limit the scope of the invention in any way.

### EXAMPLE 1

A CD19-negative relapse following a complete response to chimeric antigen receptor-modified T cells with specificity for CD19 (CART19) has been reported (Grupp et al. (2013) N. Engl. J. Med., 368:1509-1518). Briefly, a patient with pre-B-cell, acute lymphoblastic leukemia (ALL) had undergone multiple unsuccessful treatments before receiving and responding to CART19, only to relapse two months later with a disease described as CD19-negative.

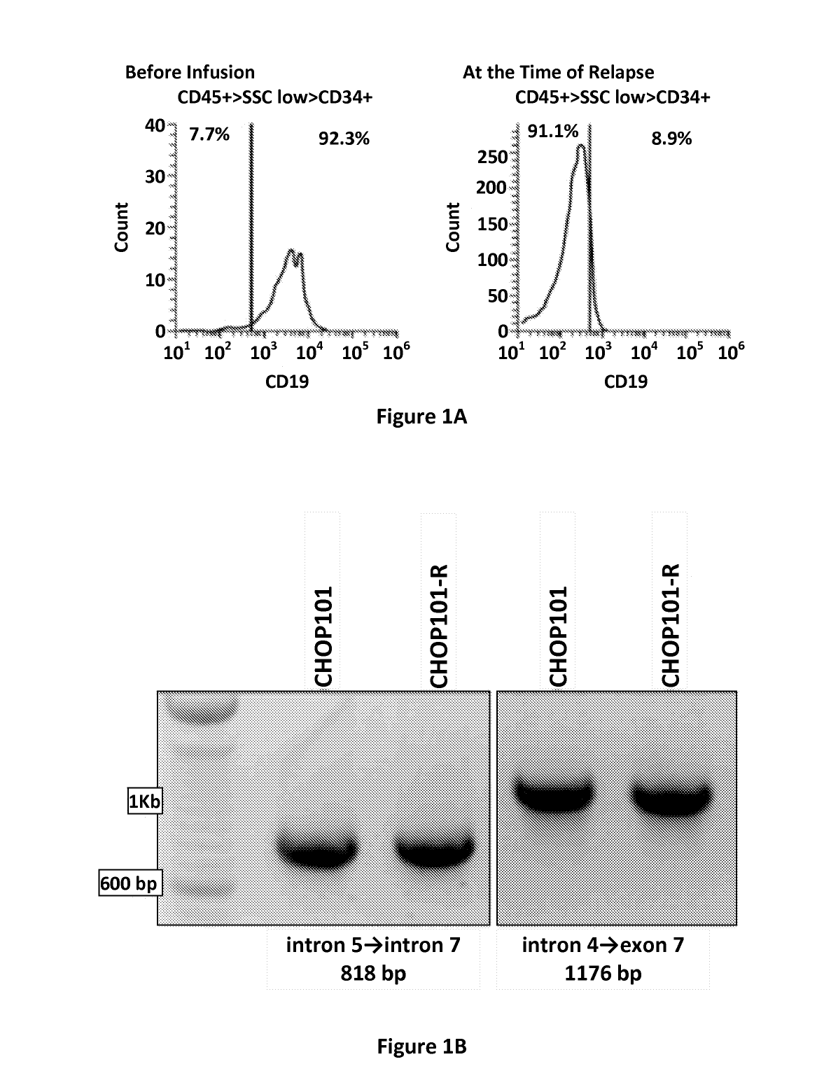

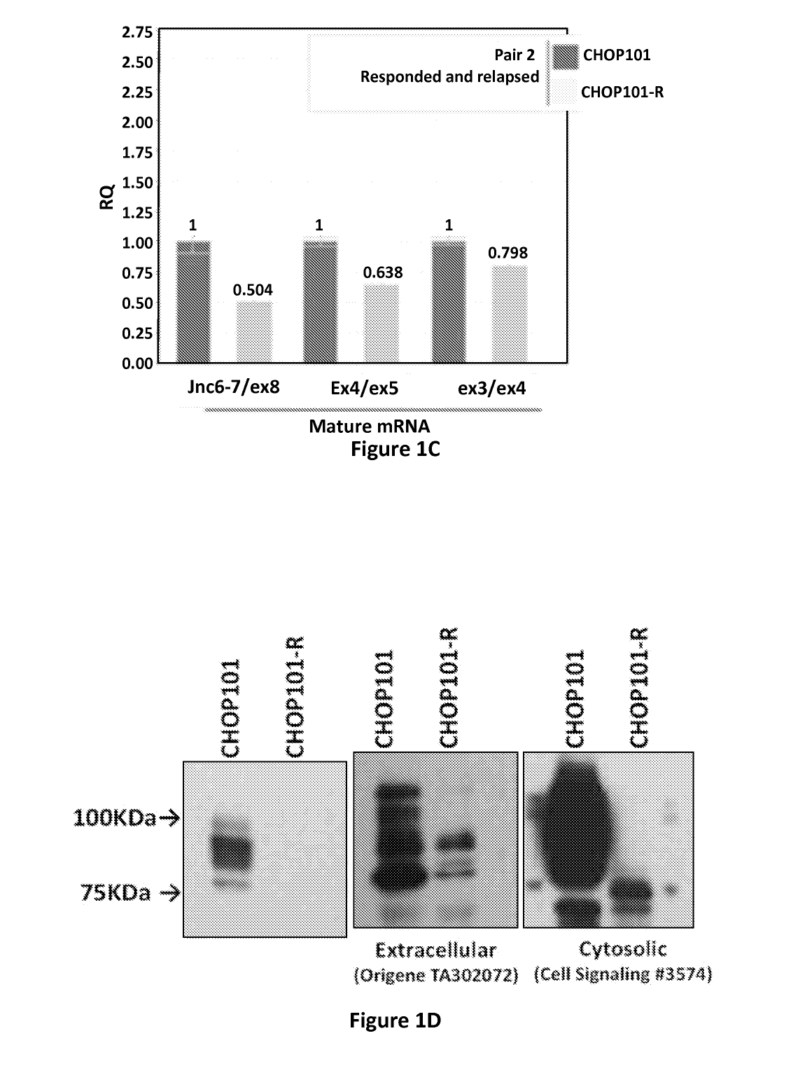

Samples from the patient were initially analyzed by flow cytometry using the same antibody that served as the backbone to make the chimeric antigen receptor (FMC63, FIG. 1A). To understand the mechanism of epitope loss, every exon and intron of the CD19 gene in the primary (CHOP101) and the relapsed (CHOP101R) leukemias were PCR-amplified and sequenced, as depicted in FIG. 1B for the “intron 4-exon 7” gene fragment. No evidence of gene deletion was observed. These data were also confirmed by whole genome sequencing. Therefore, it was hypothesized that CD19 silencing occurs at an epigenetic level. To test this hypothesis, CD19 mRNA abundance was measured using qRT-PCR with primers spanning different introns. Surprisingly, very minor down-regulation of CD19 mRNA was observed in the relapsed sample (FIG. 1C). To reconcile this result with flow cytometry data, Western blotting was performed with different antibodies. While some antibodies failed to detect CD19 protein expression in CHOP101R, others yielded several bands of lower molecular weights (TA3020072 and 3574 in FIG. 1D). Accordingly, it was concluded that the apparent down-regulation of CD19 expression in CHOP101R leukemia is likely to be post-transcriptional.

To assess the prevalence of this post-transcriptional regulation, several additional samples were xenografted. Despite the impressive successes of CART19 therapy, there have been 5 documented relapses with selective loss of CD19 expression, as measured by flow cytometry with the FMC63 antibody. Additionally, there have been reports of resistance to the CD19-CD3 bispecific antibodies (blinotumomab). Specifically, patient CHOP105 had undergone unsuccessful T-cell engraftment and relapsed quickly with CD19-positive disease (CHOP105R1). This was followed up with another round of successful CART19 therapy, resulting in complete response but eventually CD19-negative relapse (CHOP105R2). Patient CHOP107 had undergone chemotherapy with BMT prior to CART19 therapy. This, too, resulted in a CD19-negative relapse (CHOP107R). Finally, the NIH-6614 sample represents a relapse following response to blinotumomab, with no matching pre-treatment sample available.

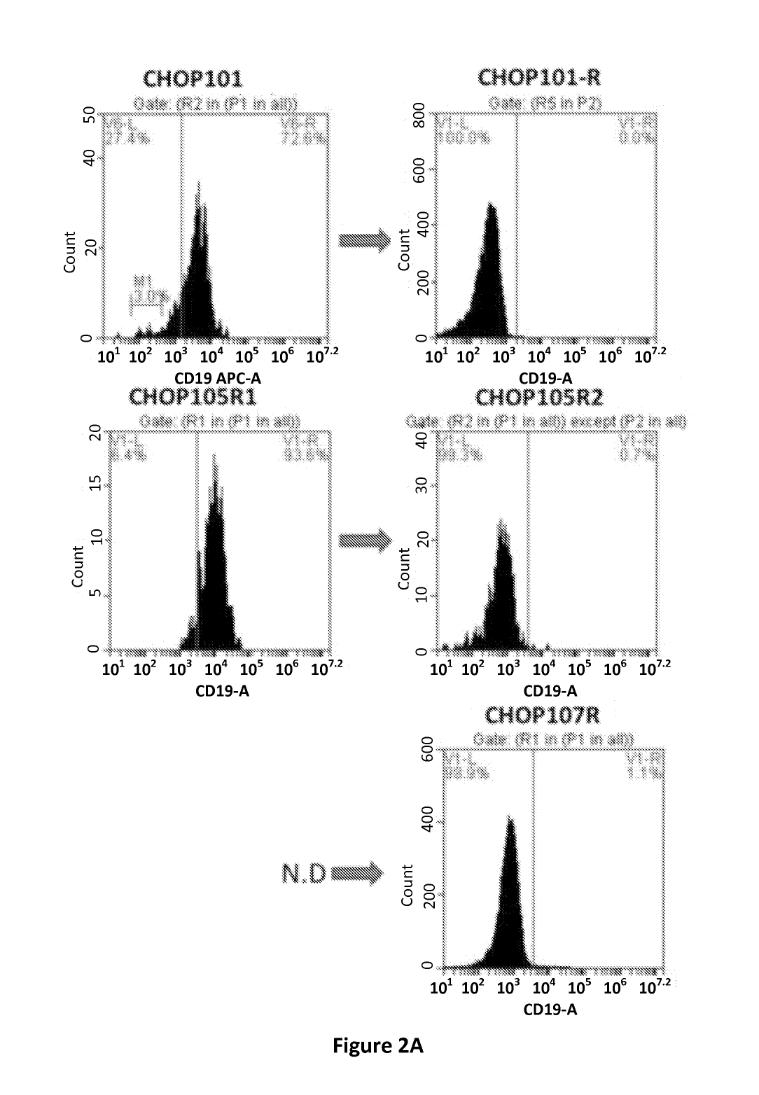

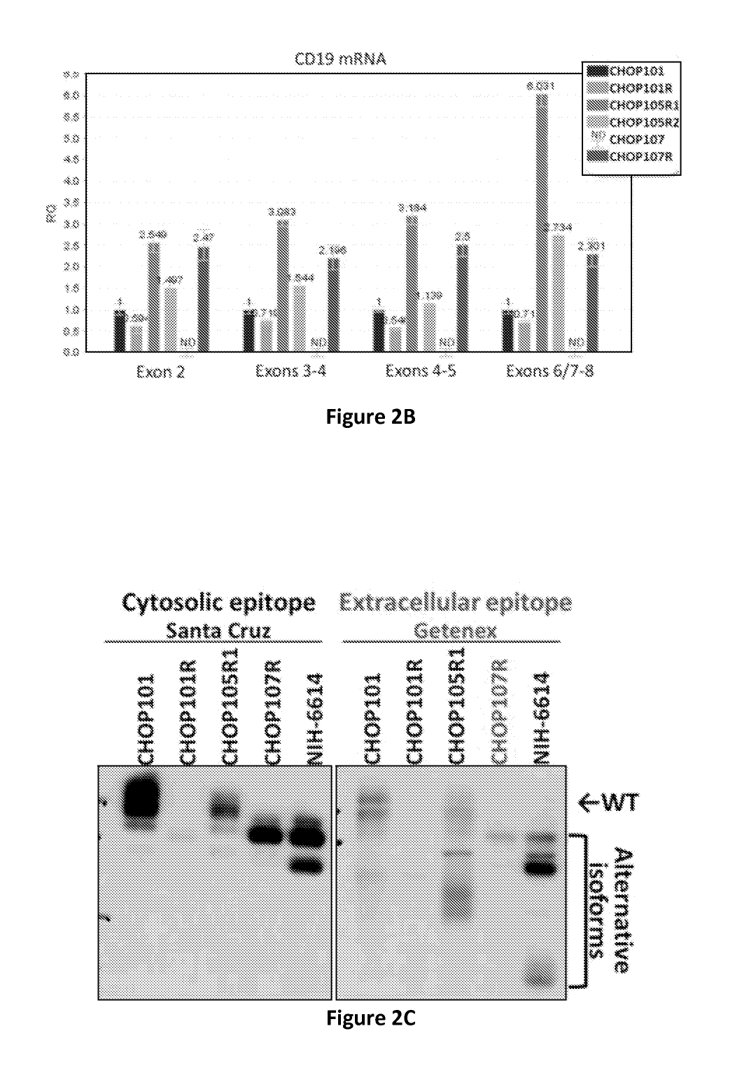

The loss of the FMC63 epitope was confirmed by flow cytometry (FIG. 2A). Robust expression of CD19 mRNA (FIG. 2B) and various protein isoforms (FIG. 2C) were observed in all FMC63-negative samples, indicating a common mechanism of CD19 deregulation, such as alternative splicing.

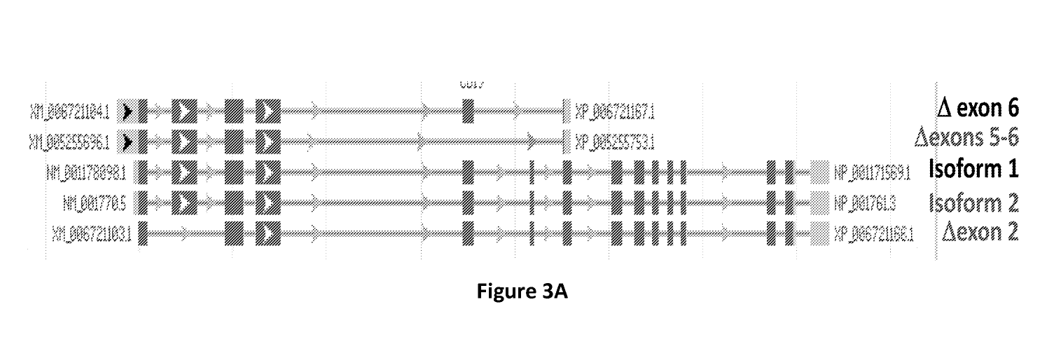

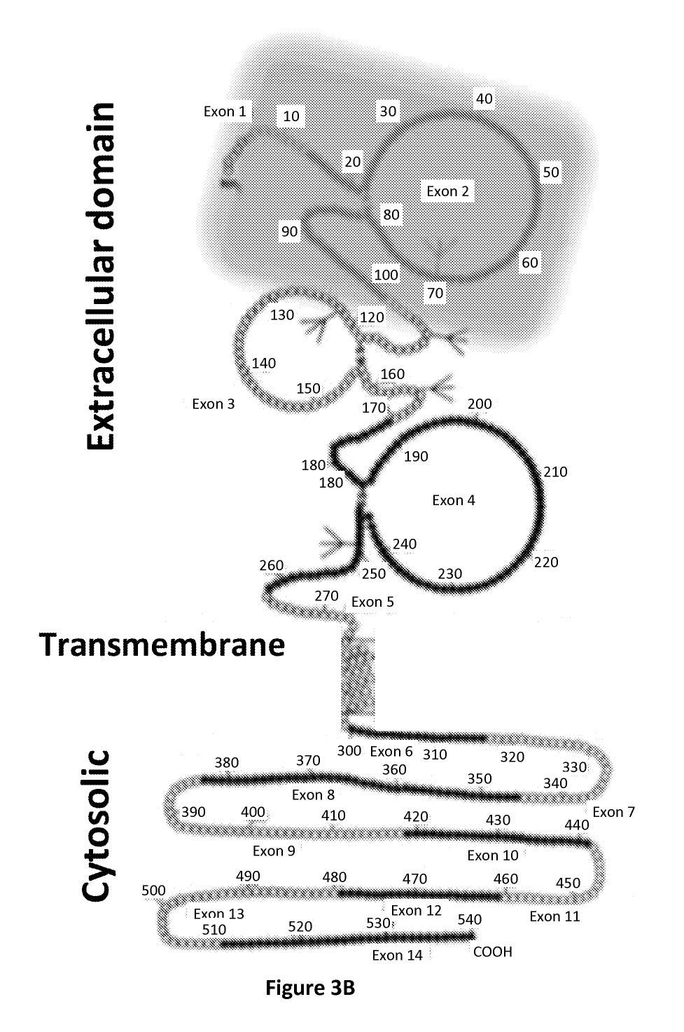

Several splice isoforms of CD19 mRNA have been included in various databases (e.g., ENSEMBL), but never validated (FIG. 3A). Notably, certain of the isoforms eliminate the FMC63 epitope encoded by exon 2 (FIG. 3B) and thereby provide a mechanism of escape.

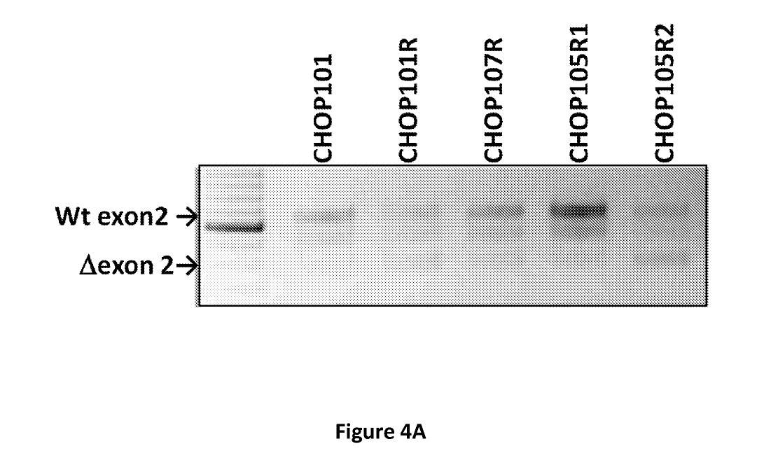

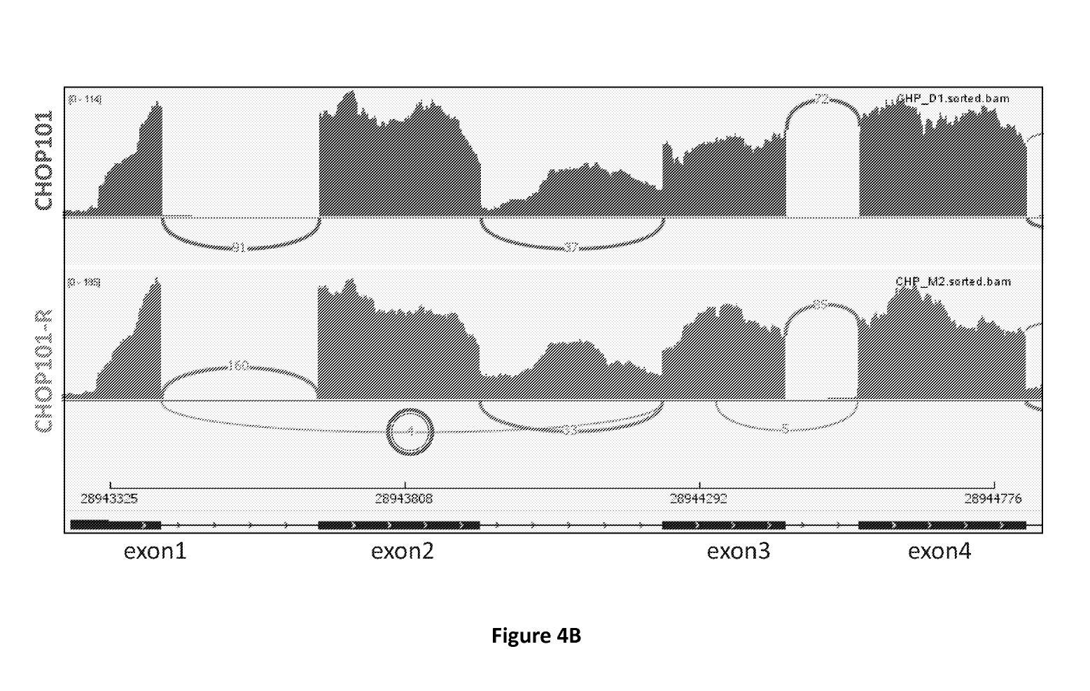

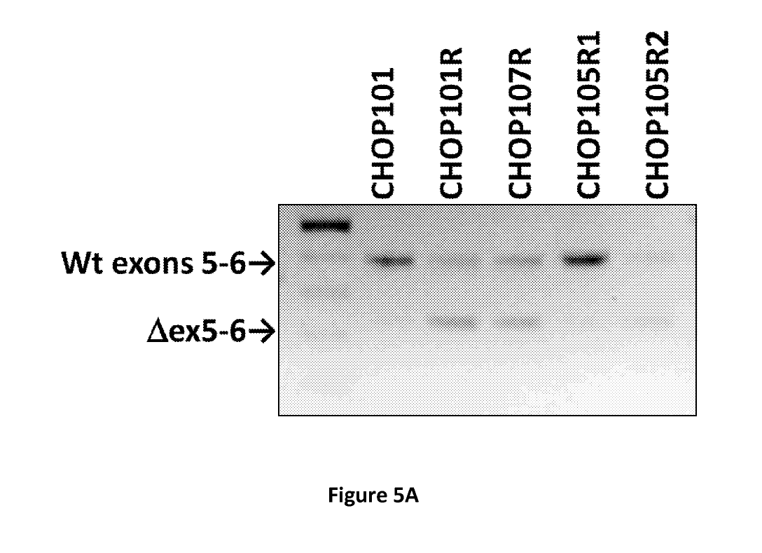

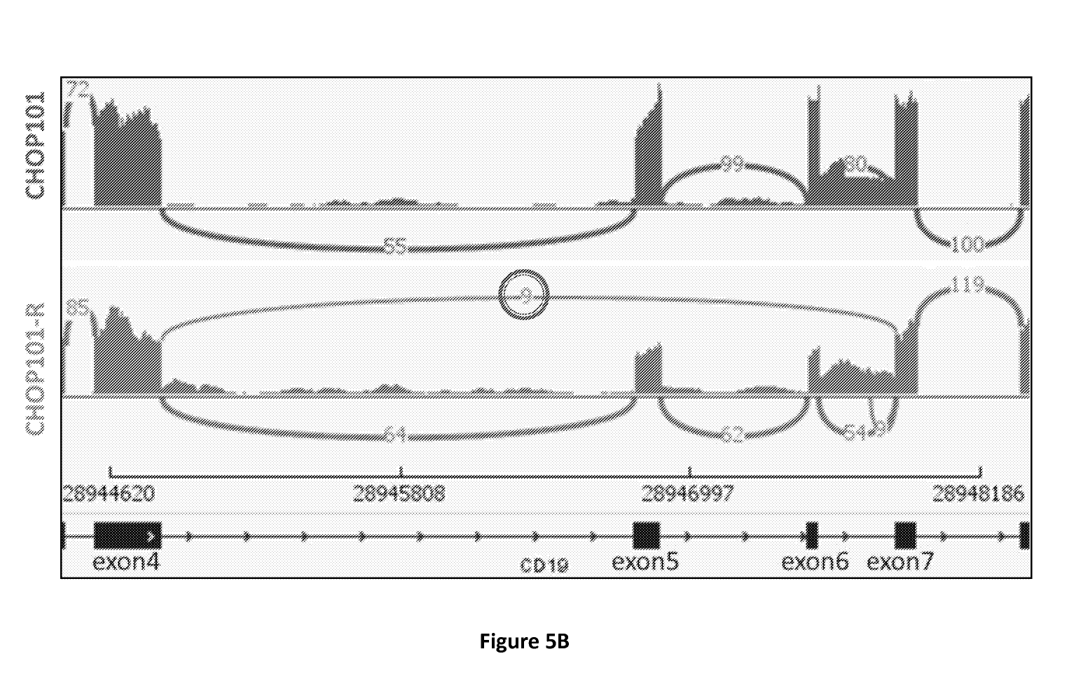

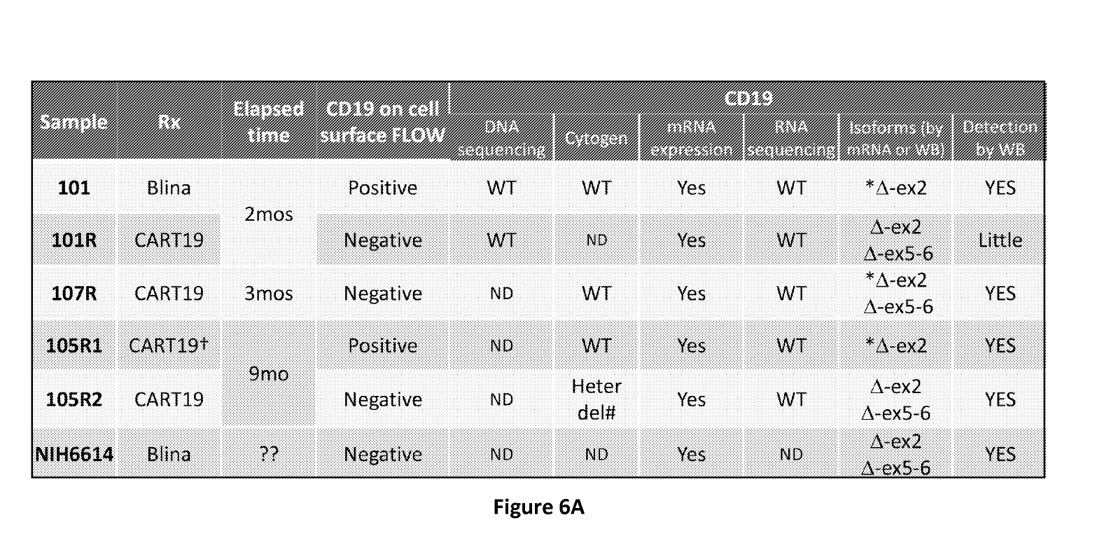

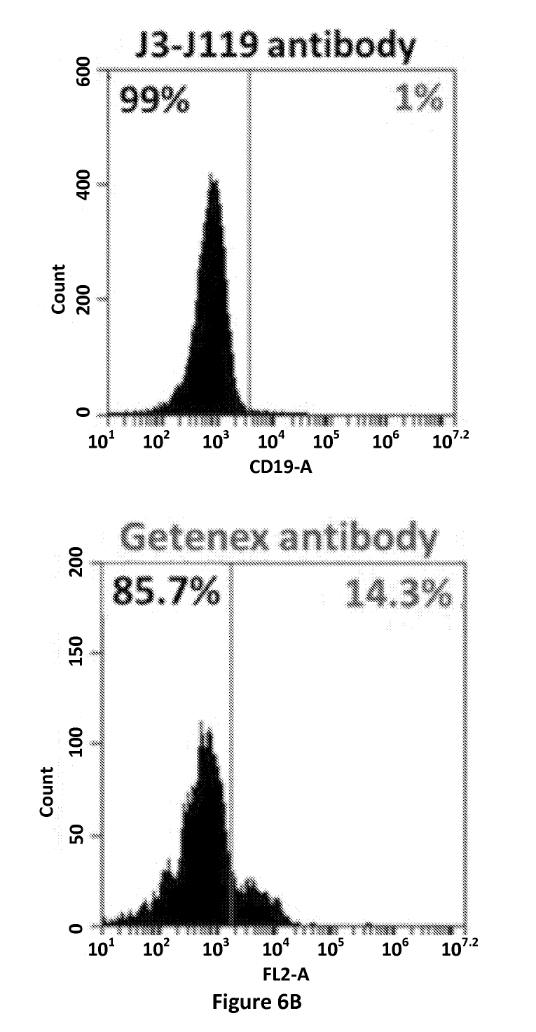

To determine whether skipping of exon 2 occurs in primary and relapsed leukemias, RT-PCR was performed on the corresponding samples. This alternatively spliced isoform was observed in all samples tested (FIG. 4A). Of note, its abundance was increased in CHOP101R vs. CHOP101 and in CHOP105R2 vs. CHOP105R1, seemingly at the expense of the full-length isoform (FIG. 4A). For the CHOP101 set, this result was corroborated using the RNASeq approach, where exon1-exon3 fusion transcripts were seen in the relapsed but not the primary leukemia (FIG. 4B). The same two approaches were used to detect exon 5-6 skipping. Once again, the abundance of this alternatively spliced transcript was increased in relapsed leukemias (FIG. 5A, 5B). The data on protein and mRNA expression in samples analyzed to date are summarized in FIG. 6A.

In summary, the data indicates that there exists a novel mechanism of resistance to immunotherapy, which is based not on mutations in the coding sequence but rather on rapid selection for alternatively spliced target protein isoforms. One important corollary of this mechanism is that post-CART19 samples may not be CD19-negative after all. Furthermore, a) remaining cytoplasmic domains can recruit and activate Lyn, thereby conferring sensitivity to inhibitors such as dasatinib, while b) remaining ectodomains, while invisible to CART19, can be targeted by other CARs and/or antibodies. Indeed, at least in the CHOP107R, surface expression of CD19 was detected using flow cytometry with one of GeneTex FACS antibodies (FIG. 6B, right panel).

### EXAMPLE 2

Despite significant advances in the treatment of pediatric B-ALL, children with relapsed or refractory disease still account for a substantial number of all childhood cancer deaths. Adults with B-ALL experience even higher relapse rates and long-term event-free survival of less than 50% (Roberts et al. (2015) Nat. Rev. Clin. Oncol., 12:344-57). Relapsed leukemia is generally not curable with chemotherapy alone, so the prospect of long-term disease control via an immunologic mechanism holds tremendous promise. One of the most innovative approaches involves the use of adoptive T cells expressing chimera antigen receptors (CAR-T) against CD19 (Porter et al. (2011) N. Engl. J. Med., 365:725-33; Kalos et al. (2011) Sci. Transl. Med., 3:95ra73). Despite obvious successes, there have been documented relapses in which CART-19 cells were still present but the leukemia cells lost surface expression of CD19 epitopes, as detected by clinical flow cytometry. According to the recent estimates, epitope loss occurs in 10-20% of pediatric B-ALL treated with CD19-directed immunotherapy (Maude et al. (2014) N. Engl. J. Med., 371:1507-17; Topp et al. (2014) J. Clin. Oncol., 32:4134-40), raising the question about its significance for neoplastic growth.

The cell surface signaling protein CD19 is required for several diverse processes in B cell development and function. In the bone marrow, CD19 augments pre-B cell receptor (pre-BCR) signaling (Otero et al. (2003) J. Immunol., 171:5921-30; Otero et al. (2003) J. Immunol., 170:73-83), thereby promoting the proliferation and differentiation of late-pro-B cells bearing functional immunoglobulin heavy chains into pre-B cells. Engaging the CD19 pathway in normal and neoplastic B-lineage cells induces the activation of the growth promoting kinases PI3K, Akt, and Lyn, which are activated via intracellular interactions with conserved tyrosine residues in the CD19 cytoplasmic tail (Wang et al. (2002) Immunity 17:501-14). Significantly, whereas CD19 possesses conserved extracellular domains needed for mature B cell function (Del Nagro et al. (2005) Immunol. Res., 31:119-31), the role of CD19 ectodomains in the proliferation and differentiation of normal B-lineage precursors is unknown. Likewise, CD19 is thought to play an essential role in B-cell neoplasm, but it is usually attributed to its ability to recruit intracellular kinases (Chung et al. (2012) J. Clin. Invest., 122:2257-66; Rickert et al. (1995) Nature 376:352-5; Poe et al. (2012) J. Immunol., 189:2318-25).

### Methods

### Cell Culture, Transfections, Treatments and Infections

All B-lymphoid cell lines (Nalm-6, Myc-5, 697 and P493-6) were cultured and maintained in RPMI 1640 medium supplemented with 10% fetal bovine serum, 2mM L-glutamine, penicillin/streptomycin at 37° C. and 5%CO₂. SMARTpool® siRNAs for splicing factors SRSF3, SRSF7, hnRNPC and hnRNPA (Dharmacon) were transfected at indicated concentrations into B-cell lines by electroporation using the AMAXA system program 0-006 and Reagent V (Lonza). siRNA knock-down efficiency was measured 24 hours and 48 hours after transfection by RT-qPCR. BCR-ligation was performed by incubation of 20×10⁶ cells with 10 μg/ml of pre-BCR specific α-IgM Jackson Immuno antibody (IgM-5μ) or with isotype control goat anti-IgG (southern biotech #0109-01) for indicated time points at RT. Cells were lysed in RIPA buffer and loaded onto PAGE gels for immunoblotting analysis. Cleavage of plasma membrane proteins by trypsin was performed by incubation of 1×10⁶ cells in 200 μl of 1× trypsin-EDTA solution (Gibco, #15400-054) in PBS for 4 minutes at 37° C. Control cells were incubated under the same conditions in PBS. Trypsinization was stopped adding 1 ml of ice cold PBS/10% FBS followed by quick centrifugation and immediate cellular lysis for whole cell protein extraction. Protein half-life was measured by treating cells with cycloheximide (Sigma) at 50 μg/mL. mRNA half-life was measured by treating the cells with Actinomycin D (Sigma) 5 μg/mL.

### Retroviral and Lentiviral Constructs

Lentiviral vector expressing luciferase and GFP pELNS-CBR-T2A-GFP has been previously described (Barrett et al. (2011) Blood 118:e112-e7). Retroviral constructs expressing full length CD19 cDNA were generated by digestion of pMX-IRES-CD19-GFP vector (Chung et al. (2012) J. Clin. Invest., 122:2257-66) with EcoRI/XhoI restriction enzymes, followed by ligation into MSCV-IRES-DsRedFP (Addgene) and pMXs-Ires-Blasticidin (RTV-016, Cell Biolabs) retroviral backbones. To generate CD19 Δex2 and CD19 Δex5-6 expressing vectors, cDNA fragments (Table 1) were synthesized (Genewiz) and cloned into MSCV-CD19-IRES-DsRedFP via EcoRI/Bg1II or Bg1II/Xhol, and later moved into pMX-IRES-Blast via EcoRI/XhoI cloning. Retroviral and lentiviral particles were generated by transfection of GP293 cells with Lipofectamine®-2000 (Invitrogen). Viral supernatants were harvested 24 hours, 36 hours, and 48 hours after transfection and used to infect B-ALL cell lines in the presence of polybrene (4 μg/ml). Where indicated, selection of infected cells was done with 10 μg/ml Blasticidine over the course of one week, or by cell sorting.

TABLE 1
DNA fragments synthesized and inserted into MSCV-CD19-IRES-
DsRedFP via EcoRI/BglII or BglII/XhoI digestion, and later moved
into pMX-IRES-Blast via EcoRI/XhoI cloning.

EcoRI/BglII CD19-Δex2 (5′→3′)
GAATTCACCACCATGCCACCTCCTCGCCTCCTCTTCTTCCTCCTCTTCCTCACCCCCATGGAAGTCAGGCCCGA
GGAACCTCTAGTGGTGAAGGTGGAAGAGGAGCTGTTCCGGTGGAATGTTTCGGACCTAGGTGGCCTGGGCTGTG
GCCTGAAGAACAGGTCCTCAGAGGGCCCCAGCTCCCCTTCCGGGAAGCTCATGAGCCCCAAGCTGTATGTGTGG
GCCAAAGACCGCCCTGAGATCT
BglII/XhoI CD19-Δex5-6 (5′→3′)
AGATCTGGGAGGGAGAGCCTCCGTGTCTCCCACCGAGGGACAGCCTGAACCAGAGCCTCAGCCAGGACCTCACC
ATGGCCCCTGGCTCCACACTCTGGCTGTCCTGTGGGGTACCCCCTGACTCTGTGTCCAGGGGCCCCCTCTCCTG
GACCCATGTGCACCCCAAGGGGCCTAAGTCATTGCTGAGCCTAGAGCTGAAGGACGATCGCCCGGCCAGAGATA
TGTGGGTAATGGAGACGGGTCTGTTGTTGCCCCGGGCCACAGCTCAAGACGCTGGAAAGTATTATTGTCACCGT
GGCAACCTGACCATGTCATTCCACCTGGAGATCACTGCTCGGCCAGATTCTTCAAAGTGACGCCTCCCCCAGGA
AGCGGGCCCCAGAACCAGTACGGGAACGTGCTGTCTCTCCCCACACCCACCTCAGGCCTCGGACGCGCCCAGCG
TTGGGCCGCAGGCCTGGGGGGCACTGCCCCGTCTTATGGAAACCCGAGCAGCGACGTCCAGGCGGATGGAGCCT
TGGGGTCCCGGAGCCCGCCGGGAGTGGGCCCAGAAGAAGAGGAAGGGGAGGGCTATGAGGAACCTGACAGTGAG
GAGGACTCCGAGTTCTATGAGAACGACTCCAACCTTGGGCAGGACCAGCTCTCCCAGGATGGCAGCGGCTACGA
GAACCCTGAGGATGAGCCCCTGGGTCCTGAGGATGAAGACTCCTTCTCCAACGCTGAGTCTTATGAGAACGAGG
ATGAAGAGCTGACCCAGCCGGTCGCCAGGACAATGGACTTCCTGAGCCCTCATGGGTCAGCCTGGGACCCCAGC
CGGGAAGCAACCTCCCTGGGGTCCCAGTCCTATGAGGATATGAGAGGAATCCTGTATGCAGCCCCCAGCTCCGC
TCCATTCGGGGCCAGCCTGGACCCAATCATGAGGAAGATGCAGACTCTTATGAGAACATGGATAATCCCGATGG
GCCAGACCCAGCCTGGGGAGGAGGGGGCCGCATGGGCACCTGGAGCACCAGGTGATCCTCAGGTGGCCAGCCTG
GATCTCCTCGAG

### Crispr/Cas9 Genome Editing System

CD19-CRISPR/Cas9-KO plasmid was obtained from Santa Cruz Biotechnologies (sc-400719) and transfected into Nalm-6 and 697 cell lines via electroporation using the AMAXA® system program 0-006 and reagent V (Lonza). Cells were stained with α-CD19-PE conjugated antibody (Pierce) 4 days after transfection, and CD19 deficient (ΔCD19) cells were sorted and plated individually in 96 well-clusters for single cell clone selection and expansion, or maintained as a pool. CD19 knock-down was confirmed by flow cytometry and by western blot using antibodies that recognize epitopes in the cytosolic and the extracellular domains of CD19. DNA and RNA were extracted and CD19 gene was sequenced to analyze the mutations induced by the CRISPR/Cas9 system. To generate frameshift mutations into CD19-exon2, a single CRISPR/Cas9 exon 2-gRNA plasmid was transfected by electroporation into 697, Nalm-6 and Raji cell lines as described above. Effective insertion of frameshift mutations at expected targeted region was assessed by Sanger sequencing.

Immunofluorescence and Colocalization Studies 697 ΔCD19 cells expressing CD19-GFP and CD19 Δex2-GFP fusion proteins incubated and stained with 5ug/ml wheat germ agglutinin Alexa Fluor®-680 (Molecular Probes, cat# W32465) by following manufacturer's instructions. Once fixed cells were mounted on a pre-charged glass microscope slides with DAPI containing medium (Vectashield, cat#H1200) and visualized under a Leica STED 3× super-resolution confocal system HC PL APO CS2 63×/1.40 Oil 63× objective. Images were acquired with using a 4184×4184 resolution with limited signal saturation. Colocalization was quantified by Pearson's correlation coefficient. Six images for each CD19 construct containing 100 cells on average were analyzed with BioImageXD and FIJI Coloc2 plugin software. Statistical Costes P-value=1 for this analysis (Costes et al. (2004) Biophys. J., 86:3993-4003).

### Cell Proliferation Assays

Myc5 cells expressing CD19-FL, CD19-Δex2 or empty Blasticidine vector, were seeded in 10mL of medium at 100,000 cells/mL. Daily samples were taken, counted by flow cytometry and cell density was calculated based on absolute counts. Each cell line was assayed in triplicates and each assay was repeated two times. 697 ΔCD19 cells expressing CD19-FL, CD19-Δex2 or CD19-Δex5-6 vector together with pELNS-CBR-T2A-GFP, were seeded in triplicate in a standard 96 well plate, 10,000 cells per well in 100 uL of media. GFP fluorescent signal was measured at 485nM excitation and 528nM emission in a Synergy™2 (Biotek) plate reader daily for 4 days. Each cell line was assayed in triplicates and each assay was repeated two times. Proliferation rates were statistically compared by Student-T test at indicated times points with *p≦0.05 and **p<0.01.

### Xenografted Tumor Samples

Xenograft models of tumor samples have been described (Barrett et al. (2011) Blood 118:e112-e7).

### Cytotoxicity Assays

Nalm-6 ΔCD19 cells expressing CD19-FL, CD19-Δex2, CD19-Δex5-6 or empty vector together with pELNS-CBR-T2A-GFP were used as targets for T cell cytotoxicity assay as described (Gill et al. (2014) Blood 123:2343-54). Briefly, target cells (T) were incubated with effector (E) T cells (CART19) at the indicated E:T ratios for 24 hours. D-luciferin (Goldbio, St. Louis, MO., cat. N. LUCK-1G) was then added to the cell culture and bioluminescence imaging was performed on a Xenogen IVIS-200 Spectrum camera. Target killing was analyzed using the software Living Image 4.3.1 (Caliper LifeSciences, Hopkinton, Mass.).

### Flow Cytometry

Live cells were stained with PE-conjugated CD19 antibody (IM1285U, Beckman Coulter) and analyzed in an Accuri™ C6 cytometer as described (Chung et al. (2012) J. Clin. Invest., 122:2257-66; Grupp et al. (2013) N. Engl. J. Med., 368:1509-18).

DNA Extraction and Sequencing of CD19 gene

Genomic DNA was obtained from 2×10⁶ cells from xenografted pre-CART and post-CART tumor samples using DNeasy® blood & tissue Kit (Qiagen). CD19 gene, expanding 1.2Kb upstream to include the enhancer and promoter regions, was amplified by PCR and sequenced. Primer sets are provided in Table 2.

TABLE 2
Sets of primers used for PCR amplification and
Sanger sequencing of CD19 gene and the
upstream-1.2 Kb enhancer and promoter regions.

| Region | | |
|---|---|---|
| CD19 gene | Direction | Sequence (5′ to 3′) |
| Enhancer | Fwd | GCAGGGGAATGACATGCTCT |
| | Rev | ATTAGCCCAGTGTCCAGCAC |
| Promoter | Fwd | ATGATGTGCTGGACACTGGG |
| | Rev | TCCCTGCCACGCTGTTTTAT |
| Promoter-Exon2 | Fwd | TCTACTCCAAGGGGCTCACA |
| | Rev | ACTGCAGCACAGCGTTATCT |
| Exon1-exon4 | Fwd | GGAGAGTCTGACCACCATGC |
| | Rev | GGACACAGAGTCAGGGGGTA |
| Exon3-intron4 | Fwd | GAGCCCCAAGCTGTATGTGT |
| | Rev | GAAGTTGGGGTTTGGGGTGA |
| Intron4 | Fwd | TTCCCTCGCTTCCAAGACAC |
| | Rev | GGGATTGTCACAGACCCTGG |
| Intron4-Intron5 | Fwd | TGCCAGGGTCTGTGACAATC |
| | Rev | AGGCTAGAGGAAGACTGGGG |
| Intron5-Intron6 | Fwd | CCCCAGTCTTCCTCTAGCCT |
| | Rev | GAAGAGGAAGTGCACGGTGA |
| exon6-exon8 | Fwd | AATGACTGACCCCACCAGGA |
| | Rev | TGCTCGGGTTTCCATAAGAC |
| Intron7-Intron12 | Fwd | GAACTTCGCCCCAGAACTGA |
| | Rev | AAGCTGCAGAGTAAGCTGGG |
| exon12-exon15 | Fwd | CAGCCGGGAAGCAACCT |
| | Rev | ATTGCTCCAGAGGTTGGCAT |

### Methylation of Promoter and Enhancer Region

Genomic DNA from xenografted tumor samples was subjected to bisulfate conversion using the Epitect Fast DNA Bisulfite kit (Qiagen). CD19 enhancer and promoter regions, as well as coding region comprising exon1-intron1-exon2-intron2, were PCR amplified using bisulfite specific primer (Table 3). PCR products were purified (PCR-purification Kit, Quiagen) and Sanger sequenced. The HOXA3 locus was used as positive control.

TABLE 3
Sets of primers for the analysis of
CpG-Methylation status after bisulfite
modification of genomic DNA.

| Region | Direction | Sequence (5′ to 3′) |
|---|---|---|
| CD19 | Fwd | AATTTTTGTTTTTAAAGGATTTTTT |
| Enhancer 1 | Rev | CTACAAATAACAAAACCTCCACTTC |
| CD19- | Fwd | TTGGGTTTTTTTAAAATAATTTTTTTT |
| Enhancer 2 | Rev | AACAAAAACCAAACACAATAATACAC |
| CD19- | Fwd | AGTTTTATTTTGGTGTTTAGGTTGG |
| Promoter 1 | Rev | AAACACATAAACTCCTTCTCAAAAA |
| CD19- | Fwd | TTTGTGTAGAAAATAGAAATGAATAAATAA |
| Promoter 2 | Rev | AACAAAAACCTAAAAAACACTCAAC |
| CD19- | Fwd | GTAGATATTTATGGTTGAGTGTTTTTTAGG |
| Exon 1 | Rev | TTCCTAAATTTTACAAATAAAAAAATAAAA |
| HOXA3 | Fwd | GGTTTTTATTAGATTTTGGGGTTTT |
| | Rev | TAATATCTCTACAACCTTCCCCAAC |

### Reverse Transcription and Radioactive Semiquantitative PCR

Reverse transcription-PCR (RT-PCR) was performed as described (Lynch et al. (2000) Mol. Cell Biol., 20:70-80), using sequence-specific primers. PCR step was performed with radiolabeled primers (Table 4) and cycle numbers chosen to provide a signal that is linear with respect to input RNA. Quantification was done by densitometry using a Typhoon™ PhosphorImager (Amersham Biosciences).

TABLE 4
Sets of primers used for radioactive semiquantitative
PCR analysis of CD19 mRNA isoforms.

| Region | Direction | Sequence (5′ to 3′) | Full Length | Δex2 | Δex5-6 |
|---|---|---|---|---|---|
| Exon 1-4 | Fwd | CCGAGGAACCTCTAGTGGTGAAGG | 544 bp | 268 bp | |
| | Rev | CCACAGGACAGCCAGAGTGTGGA | | | |
| Exon 4-8 | Fwd | CCTAAGTCATTGCTGAGCCTAGAGC | 487 bp | | 327 bp |
| | Rev | CGCTGCTCGGGTTTCCATAAGACG | | | |

CD19 mini-gene Crosslinking and Pull-Down Assays

The following region expanding exon 2 and 220 nucleotides of its flanking introns, was synthesized (Genewiz) and cloned into pBSKii+ (Table 5). This mini-gene gene was transcribed in vitro, radioactively labeled with CTP-³²P and incubated with nuclear lysates from Nalm-6 B-ALL cells for 30 minutes at 30° C. Exposure to UV light (254 nm) induced covalent cross-linking of nuclear proteins to RNA (Rothrock et al. (2005) EMBO J. 24:2792-802). Immunoprecipitation of crosslinked RNA/protein complexes using antibodies specific for splicing factors (Table 6) was performed as described (Lynch et al. (1996) Genes Dev., 10:2089-101; Mallory et al. (2011) Mol. Cell Biol., 31:2184-95).

TABLE 5
Sequence of the mini-gene expanding exon2 and 220nt of its
flanking introns that was synthesized (Genewiz) and inserted
into pBSKii + via XhoI/NotI cloning.

XhoI/NotI CD19-int1-ex2-int2(5′→3′)
CTCGAGGAAGGGATTGAGGCTGGAAACTTGAGTTGTGGCTGGGTGTCCTTGGCTGAGTAACTTACCCTC
TCTGAGCCTCCATTTTCTTATTTGTAAAATTCAGGAAAGGGTTGGAAGGACTCTGCCGGCTCCTCCACT
CCCAGCTTTTGGAGTCCTCTGCTCTATAACCTGGTGTGAGGAGTCGGGGGGCTTGGAGGTCCCCCCCAC
CCATGCCCACACCTCTCTCCCTCTCTCTCCACAGAGGGAGATAACGCTGTGCTGCAGTGCCTCAAGGGG
ACCTCAGATGGCCCCACTCAGCAGCTGACCTGGTCTCGGGAGTCCCCGCTTAAACCCTTCTTAAAACTC
AGCCTGGGGCTGCCAGGCCTGGGAATCCACATGAGGCCCCTGGCCATCTGGCTTTTCATCTTCAACGTC
TCTCAACAGATGGGGGGCTTCTACCTGTGCCAGCCGGGGCCCCCCTCTGAGAAGGCCTGGCAGCCTGGC
TGGACAGTCAATGTGGAGGGCAGCGGTGAGGGCCGGGCTGGGGCAGGGGCAGGAGGAGAGAAGGGAGGC
CACCATGGACAGAAGAGGTCCGCGGCCACAATGGAGCTGGAGAGAGGGGCTGGAGGGATTGAGGGCGAA
ACTCGGAGCTAGGTGGGCAGACTCCTGGGGCTTCGTGGCTTCAGTATGAGCTGCTTCCTGTCCCTCTAC
CTCTCACTGTCTTCTCTCTCTGCGGGTCTTTGTCTCTATTTATCTCTGTGCGGCCGC

TABLE 6
Antibodies used for pull down assays for the identification of splicing
factors that bind to the CD19-int1-exon2-int2 mini-gene.

| Antibody | Company | Catalog # |
|---|---|---|
| PTB | Calbiochem | NA63 |
| NOVA1 | Abcam | ab77594 |
| HuR/D | Santa Cruz | sc-5261 |
| PSF | Sigma | P2860 |
| Celf1 | Novus | NB200-316 |
| Celf2 | University of Florida | |
| hnRNP-A1 | Abcam | ab5832 |
| hnRNP-C1/C2 | Abcam | ab10294 |
| hnRNP-F/H | Abcam | ab10689 |
| hnRNP-K | Abcam | ab39975 |
| hnRNP-L | Abcam | ab6106 |
| hnRNP-U | Abcam | ab10297 |
| SRSF2 | Sigma | S4045 |
| SRSF3 | Thermo/Life Tech | 33-4200 |
| SRSF6 | Aviva | ARP40632 |
| SRSF7 | MBL | RN079PW |

### Western Blotting and Coimmunoprecipitation

Whole-cell protein lysates were obtained in RIPA buffer. Protein concentrations were estimated by Biorad colorimetric assay. Immunoblotting was performed as previously described by loading 10 μg of protein onto 7.5% PAGE gels (Sotillo, et al. (2011) Oncogene 30:2587-94). Signals were detected by ECL (Pierce) or by Odyssey Infrared Imager (LI-COR Biosciences). Representative blots are shown. Antibodies used are listed in Table 7. Coimmunoprecipitations were performed in whole-cell protein lysates from 15 million cells in 500 μL of nondenaturing buffer (150 mmol/L NaCl, 50 mmol/L Tris-pH8, 1% NP-10, 0.25% sodium deoxycholate) and 10 μL of kinase-specific antibodies. After overnight incubation at 4° C., 50 μL of Protein A agarose beads (Invitrogen) were added and incubated for 1 hour at 4° C. Beads were washed 3 times with nondenaturing buffer, and proteins were eluted in Laemmli sample buffer, boiled, and loaded onto PAGE gels.

TABLE 7
Primary and secondary antibodies used for immunoblotting
and immunoprecipitations after protein separation by
SDS-PAGE and transference to PVDF membrane.

| Protein | Company | Clone/Catalog# | Epitope |
|---|---|---|---|
| CD19 | Origene | 3B10/TA506236 | N′terminus |
| | Genetex | 5F3/GTX84726 | N′terminus |
| | Cell signaling | #3574 | C′terminus |
| | Santa Cruz | LE-CD19 | C′terminus |
| AKT | Cell signaling | #4691 | N/A |
| P-AKT-S473 | Cell signaling | #4060 | N/A |
| SRSF3 | Thermo/Life Tech | 33-4200 | N/A |
| Actin | Sigma | A3853 | N/A |
| Lyn | Cell Signaling | #4576 | N/A |
| PI3K | Cell Signaling | #4292 | N/A |
| goat anti-rabbit-HRP | GE Healthcare | NA934V | N/A |
| goat anti-mouse-HRP | GE Healthcare | NA931V | N/A |
| goat anti-mouse-800 | LICOR | 926-32210 | N/A |
| goat anti-rabbit-680 | LICOR | 827-11081 | N/A |

N/A: Not applicable.

### Deglycosylation Assay

Whole cell protein lysates were obtained using a non-denaturing buffer (150mM NaCl, 50mM Tris-pH8, 1% NP-10, 0.25% Sodium deoxycholate) and treated with deglycosylation mix (New England Biolabs, #P6039S) following manufacturer's instructions in the presence of protease and phosphatase inhibitors (Thermo Scientific #78446). Control and deglycosylated lysates were loaded onto 8% PAGE gels for western blot analysis.

### Reverse Transcription, Real Time-Quantitative PCR (RT-qPCR) and PCR

Total RNA was isolated using TRIzol® reagent (Invitrogen). cDNAs were prepared with random hexamers using High Capacity cDNA RT kit (Life Technologies). CD19 mRNA isoforms were visualized in 1% agarose gels after semiquantitative PCR amplification of cDNA using Platinum Taq-polymerase (Invitrogene) following manufacturer's instructions. Primers used for each CD19 isoform and expected amplicon sizes are listed in Table 8. When required, individual bands were gel-purified (QlAquick® Gel Extraction Kit, Qiagen), and Sanger sequenced. RT-QPCR was performed using Power SYBR® Green PCR Master Mix (Life Technologies) and gene-specific oligo pairs (Table 9). Reactions were performed on an Applied Biosystems Viia7 machine and analyzed with Viia7 RUO software (Life Technologies).

TABLE 8
Pairs of oligos for semiquantitative PCR amplification of CD19
cDNA followed by visualization in agarose gel. Same primers were
used for sequencing after gel purification of specific bands.

| | | | | Full | | |
|---|---|---|---|---|---|---|
| | Region | Dir. | SEQUENCE (5′→3′) | Length | Δex2 | Δex5-6 |
| CD19 cDNA PCR | Exon1-4 | Fwd | GGAGAGTCTGACCACCATGC | 640 bp | 374 bp | |
| | | Rev | GGACACAGAGTCAGGGGGTA | | | |
| | Exon4-8 | Fwd | AAGGGGCCTAAGTCATTGCT | 490 bp | | 331 bp |
| | | Rev | TGCTCGGGTTTCCATAAGAC | | | |
| | Exon1-5 | Fwd | GGCCCGAGGAACCTCTA | 800 bp | 533 bp | no |
| | | Rev | CAGCAGCCAGTGCCATAGTA | | | amplif |

TABLE 9
Pairs of oligos used for Real
Time-qPCR amplification of cDNA.

| Region | Direction | Sequence (5′ to 3′) |
|---|---|---|
| SRSF3 | Fwd | CACCCGGCTTTGCTTTTGTT |
| | Rev | CGGCAGCCACATAGTGTTCT |
| SRSF2 | Fwd | CGGAGCCGCAGCCCTA |
| | Rev | GGTCGACCGAGATCGAGAAC |
| hnRNPC | Fwd | AGAACCCGGGAGTAGGAGAC |
| | Rev | AGCCGAAAATGTAGCTGAAGA |
| hnRNPA1 | Fwd | GGTAGGCTGGCAGATACGTT |
| | Rev | TAACGATGCTTCTTCGGCGG |
| Actin | Fwd | AGCATCCCCCAAAGTTCAC |
| | Rev | AAGGGACTTCCTGTAACAACG |
| CD19 | Fwd | GGAGAGTCTGACCACCATGC |
| Ex1-ex2 | Rev | ACTGCAGCACAGCGTTATCT |
| CD19 | Fwd | GAGCCCCAAGCTGTATGTGT |
| Ex3-ex4 | Rev | GGACACAGAGTCAGGGGGTA |
| CD19 | Fwd | GCCTCCTCTTCTTCCTCCTCTT |
| Jnct Ex1/3 | Rev | CCGGAACAGCTCCCCTTCCACCTTC |
| CD19 | Fwd | AAGGGGCCTAAGTCATTGCT |
| Ex4-ex5 | Rev | CAGCAGCCAGTGCCATAGTA |
| CD19 | Fwd | CCCCACCAGGAGATTCTTCA |
| Jnct Ex6/7-ex8 | Rev | TGCTCGGGTTTCCATAAGAC |

### RNA-seq

RNA-sequencing reads were aligned using STAR version 2.4.0b (Dobin et al. (2013) Bioinformatics 29:15-21) with a custom index based on the hg19 reference genome and a splice junction database consisting of all RefSeq isoforms supplemented with the exon 1-3 (Δex2) exon 4-7 (Δex5-6) junctions for CD19. Aligned read counts per gene were computed using the htseq-count software with “- -mode=intersection-strict” and normalized to gene RPKM by the formula: 10⁹*(read count aligning to gene)/((mRNA length in bp)*(total aligned read count over all genes)).

### WGS/WES Bioinformatic Processing and Point Mutation and LOH Analysis

Read alignment to the hg19 human reference genome for whole exome and whole genome sequencing samples was performed using the bwa v0.7.7 algorithm with default parameters. Unbiased point mutation calling was performed using samtools and bedtools v0.1.18, and the aligned sequence at CD19 was further manually reviewed in the Integrative Genomics Viewer (IGV) in order to detect subclonal mutations in CD19. For genome wide loss of heterozygosity (LOH) analysis based on WES samples, B allele fractions (BAF) were computed for all common germline SNPs in dbSNP build 142 (obtained from the UCSC Genome Browser) using samtools mpileup v0.1.18. Genomic BAF profiles were then visualized in the R statistical programming language.

### AVISPA Splicing Predictions

To find putative regulators of CD19 exon 2 and exon 5-6 skipping, AVISPA (avispa.biociphers.org) was used, which predicts not only if a cassette exon is alternatively spliced, but also gives a list of putative regulatory motifs that contribute to this splicing outcome (Barash et al. (2013) Genome Biol., 14:R114). Hg19 coordinates were extracted for exons 1 through 3 to define the exon 2 triplet. Because AVISPA currently only handles single cassette exon events as inputs, coordinates for exons 4 through 7 were extracted to define two overlapping cassette exon triplets for the tandem skipping of exons 5 and 6 (i.e., an exon 4, 5, 6 triplet and an exon 4, 5, 7 triplet). These three triplets were run and the top motifs and predicted associated splicing factors for the alternative versus constitutive splicing prediction step were compared. These top motifs were defined by their normalized feature effect (NFE), described in (Barash et al. (2013) Genome Biol., 14:R114). Briefly, this value represents the effect on splicing prediction outcome if a motif is removed in silico, normalized by the total effects observed from removing each of the top features in this way. This method has been used to detect and experimentally verify novel regulators of cassette exon splicing (Gazzara et al. (2014) Methods 67:3-12).

### MAJIQ and VOILA Splicing Analysis

In order to identify and visualize splicing variations in CD19 from RNA-Seq, the MAJIQ and VOILA software (Vaquero-Garcia et al. (2014) Splicing analysis using RNA-Seq and splicing code models—from in silico to in vivo. 11th Integrative RNA Biology Meeting; Boston, Mass., p. 41) were applied. Briefly, STAR (Dobin et al. (2013) Bioinformatics 29:15-21) was run to map the RNA-Seq reads. Next, MAJIQ used the junction spanning reads detected by STAR to construct a splice graph of CD19 and quantitate the percent spliced in (PSI) of the alternative exons. Finally, the VOILA visualization package was used to plot the resulting splice graph, the alternative splicing variants, and the violin plots representing the PSI estimates.

### RESULTS

Post-CART-19 Pediatric B-ALL Relapses Retain and Transcribe the CD19 gene

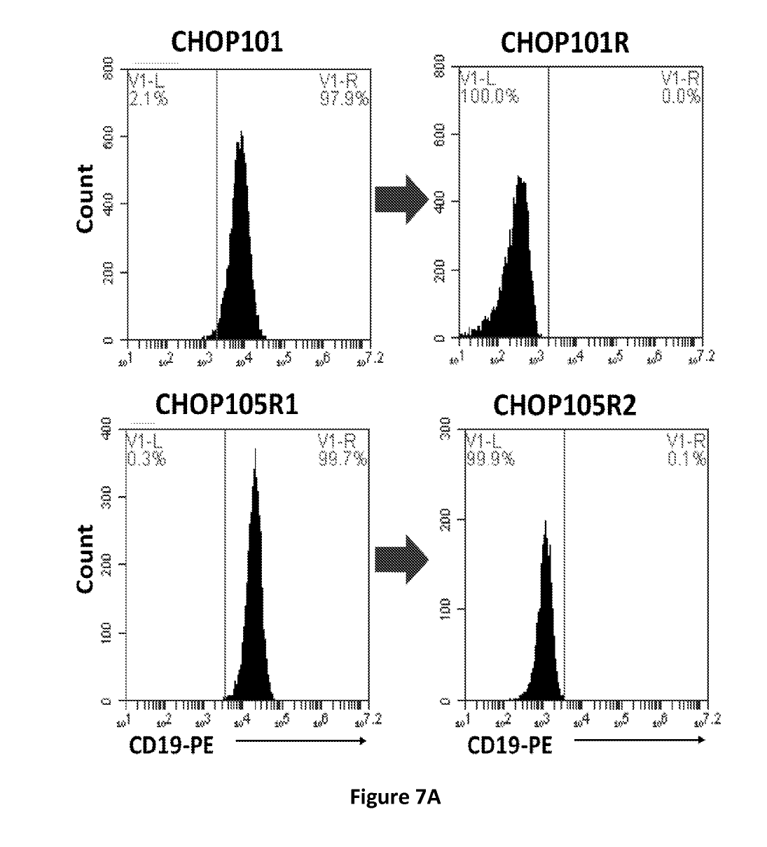

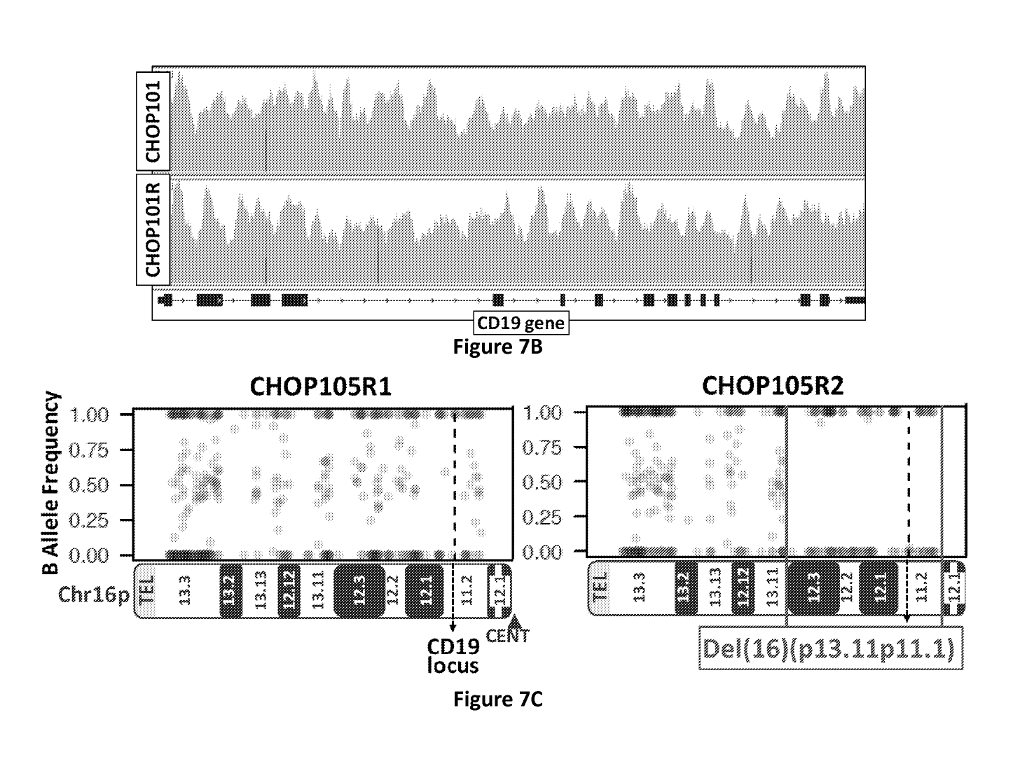

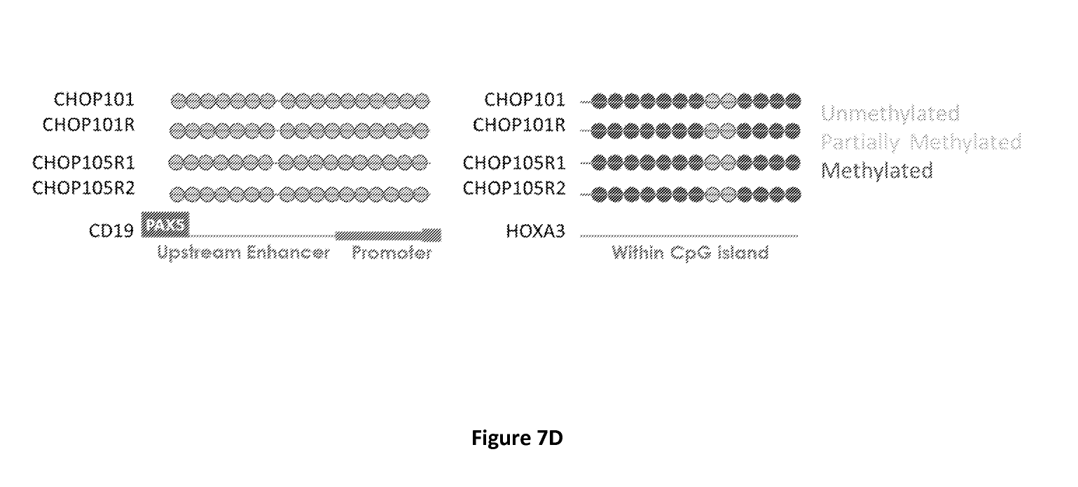

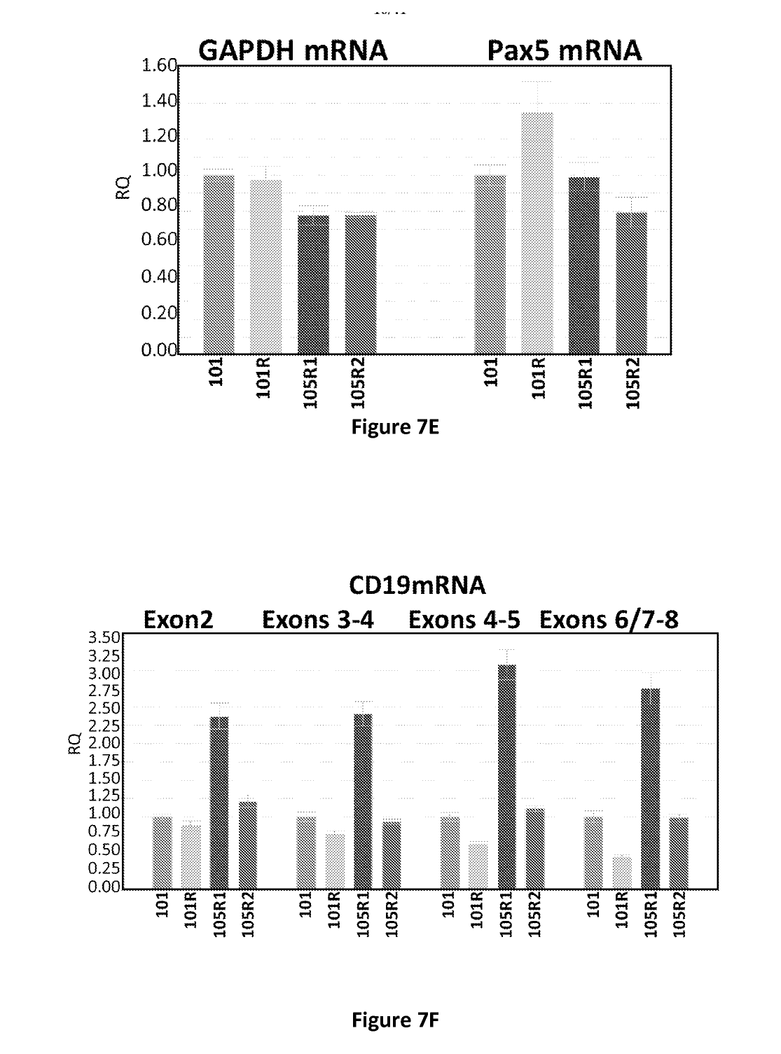

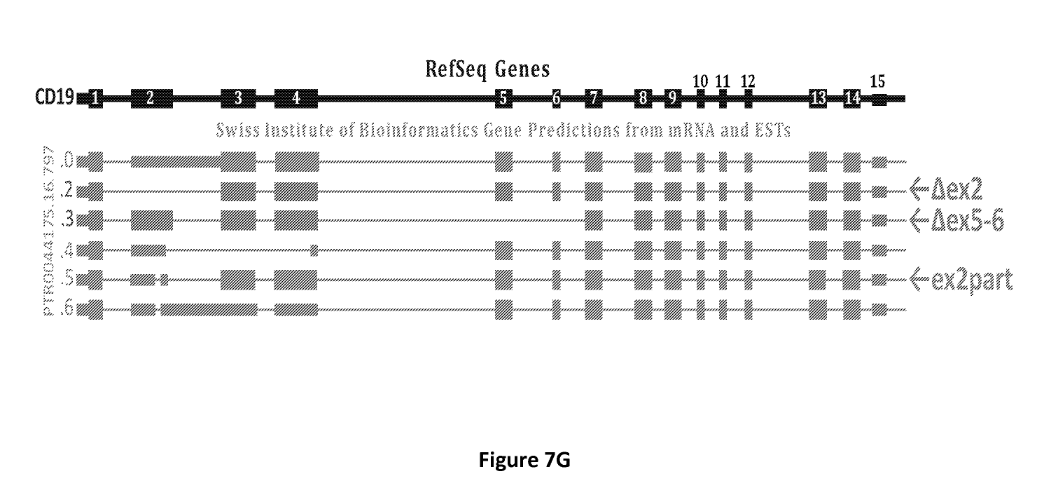

To study mechanisms and consequences of CD19 loss in vivo, the CD19-positive pre-CART-19 leukemia and the relapsed CD19-negative leukemia obtained from the same patient were analyzed (Grupp et al. (2013) N. Engl. J. Med., 368:1509-18) (CHOP101/101R in FIG. 7A, top). Two sequential relapses after CART-19 therapy from patient CHOP105 were also studied. The first CD19-positive relapse (R1) was due to the loss of CART cells, and the patient achieved complete remission following CART-19 re-infusion. However, the second relapse (R2) was accompanied by loss of the CD19 epitope (FIG. 7A, bottom) and rapid disease progression. Upon successful engraftment in NSG mice, these four paired leukemia samples were used for molecular analyses. Samples CHOP101/101R were subjected to whole genome sequencing, and no copy number variations or focal deletions in the CD19 locus were observed (FIG. 7B). Clinical karyotyping and LOH analysis of samples CHOP105R1/R2 revealed a very large hemizygous deletion within chromosome 16 extending from p13.11 to p11.1 (FIG. 7C) and spanning the entire CD19 locus.

To further characterize the B-ALL samples, whole exome (WES) and RNA sequencing was performed as well as copy number alteration (CNA) analysis. These approaches revealed the existence in relapsed leukemias of de novo genomic alterations primarily, but not exclusively, affecting exon 2. In sample CHOP101R, two independent frameshift mutations (one in exon 2 and one in exon 4) were observed. However, they were each sub-clonal and accounted for less than 50% of tumor cells. In the CHOP105 sample, the insertion of 3 codons in exon 2 were identified, which was detectable with very low frequency by RNA-Seq in the R1 leukemia but became clonal in the R2 leukemia (Table 10). To better understand the relevance of such mutations, three other post-CART-19 relapses were analyzed: CHOP107Ra/107Rb and CHOP133R, for which matched baseline samples were not available. Neither of the CHOP107R samples (which had been xenografted from the same patient at different times during disease progression) contained mutations. However, leukemia CHOP133R suffered hemizygous loss of the entire chromosome 16, and the remaining allele contained a frame shift mutation also in exon 2 (Table 10), which could have led to nonsense-mediated decay (Dreyfuss et al. (2002) Nat. Rev. Mol. Cell. Biol., 3:195-205). In summary, genetic alterations could have accounted for CD19 protein loss in some (e.g., CHOP105R2 and CHOP133R) but not in other (e.g., CHOP101R and CHOP107a/b) samples, prompting investigation into transcriptional deregulation.

Using bisulfite-based sequencing, it was shown that there was no increase in methylation of CD19 promoter or enhancer elements, which could have accounted for gene silencing in the two matched relapse samples (FIG. 7D). qRT-PCR for PAX5, the key regulator of CD19 transcription (Kozmik et al. (1992) Mol. Cell Biol., 12:2662-72), was also perofmed, but no consistent down-regulation of PAX5 mRNA was observed (FIG. 7E). More surprisingly, CD19 mRNA levels were found to be down-regulated only 2-3-fold, depending on the choice of primers (FIG. 7F). The discrepancy between mRNA and protein levels suggested that post-CART19 samples may have altered regulation of transcript processing.

Alternatively Spliced CD19 mRNA Variants Accumulate in Post-CART19 Relapses

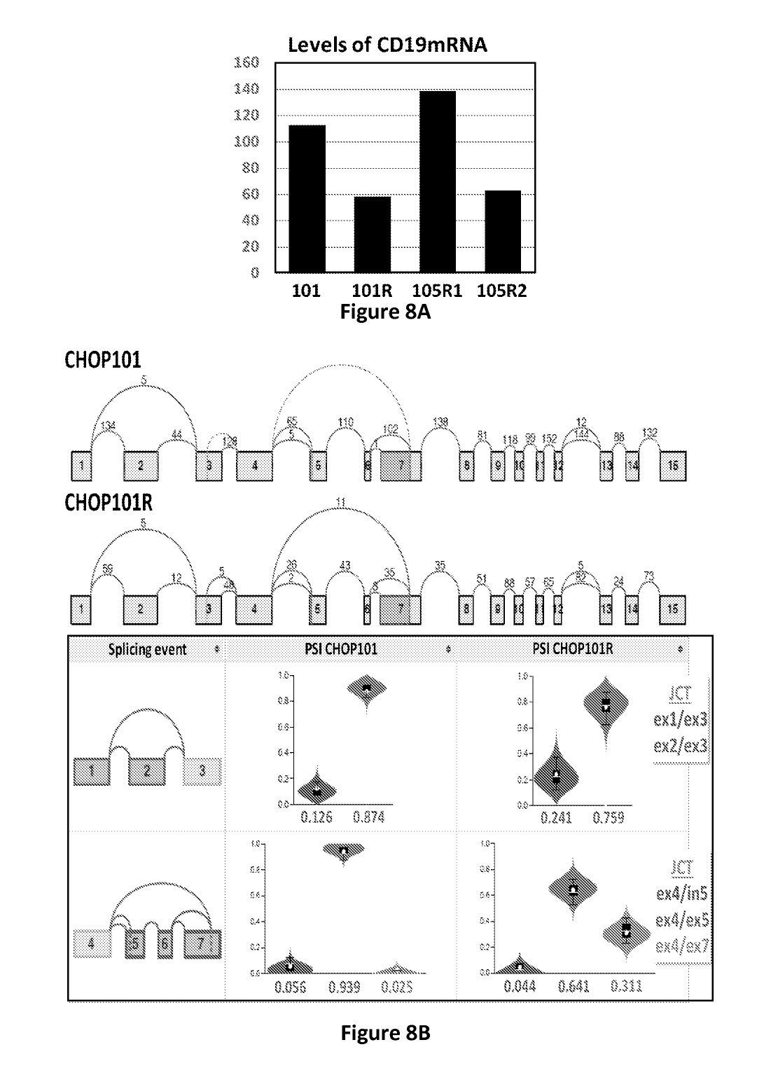

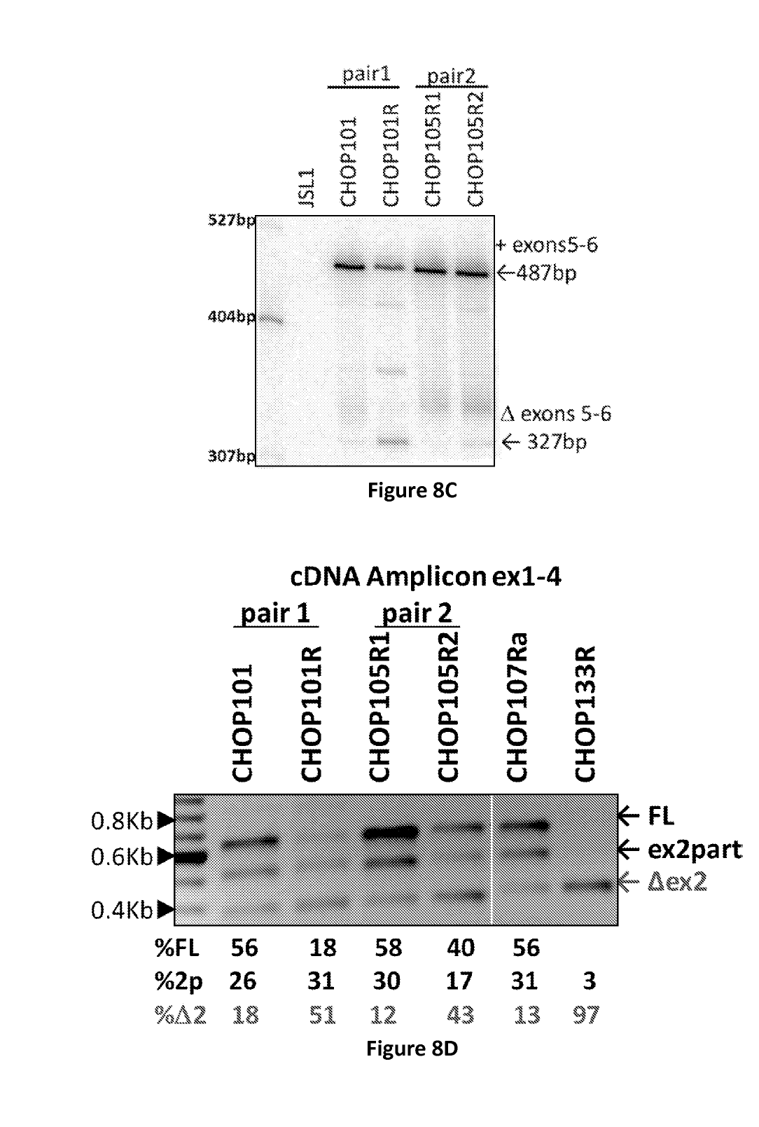

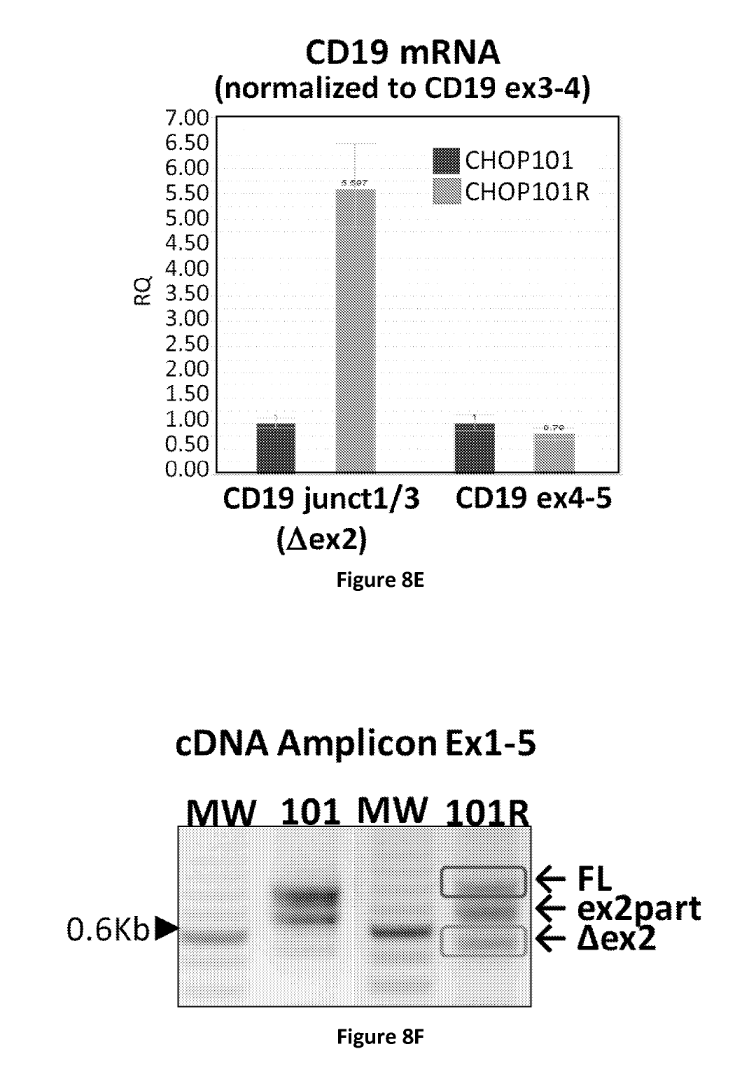

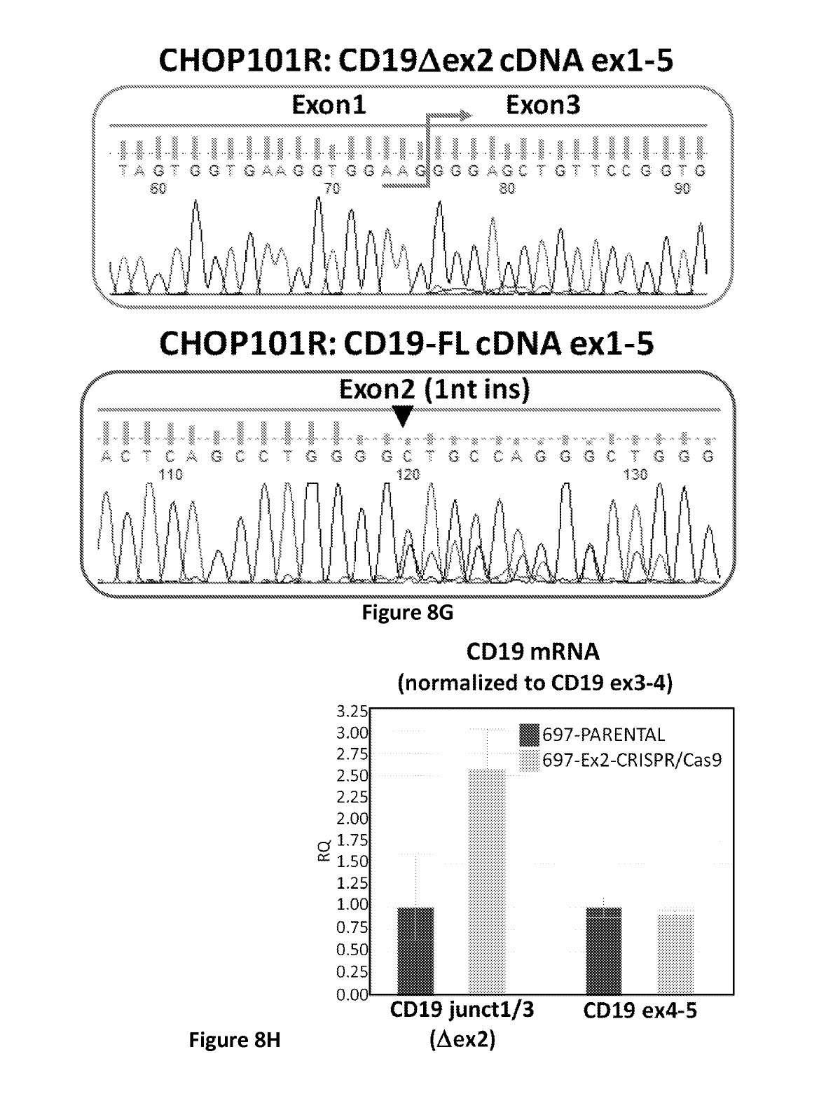

The SIB Genes Track (Benson et al. (2004) Nucleic Acids Res., 32:D23-6) implemented in UCSC Genome Browser postulates the existence of CD19 mRNA isoforms skipping exons 2 and 5-6 (FIG. 7G). To study these isoforms, sustained CD19 mRNA expression in relapsed tumors was confirmed using RNA-Seq (FIG. 8A) and then aligned CHOP101/101R RNA-Seq reads to CD19 exons using the MAJIQ algorithm (Vaquero-Garcia et al. (2014) Splicing analysis using RNA-Seq and splicing code models - from in silico to in vivo. 11th Integrative RNA Biology Meeting; Boston, Mass. p. 41) (FIG. 8B, top). These alignments were used to estimate the relative inclusion (percent spliced in, PSI) of splicing variants and visualize them in violin plots generated by VOILA (Vaquero-Garcia et al. (2014) Splicing analysis using RNA-Seq and splicing code models—from in silico to in vivo. 11th Integrative RNA Biology Meeting; Boston, Mass. p. 41) (FIG. 8B, bottom). This analysis revealed that in CHOP101 exon 4 is always spliced to exon 5, while in CHOP101R 25-30% of the observed transcripts skip exon 5-6, leading to juxtaposition of exons 4 and 7. A trend toward fewer reads spanning ex1/2 and ex2/3 junctions in CHOP101R was also observed (FIG. 8B, bottom).

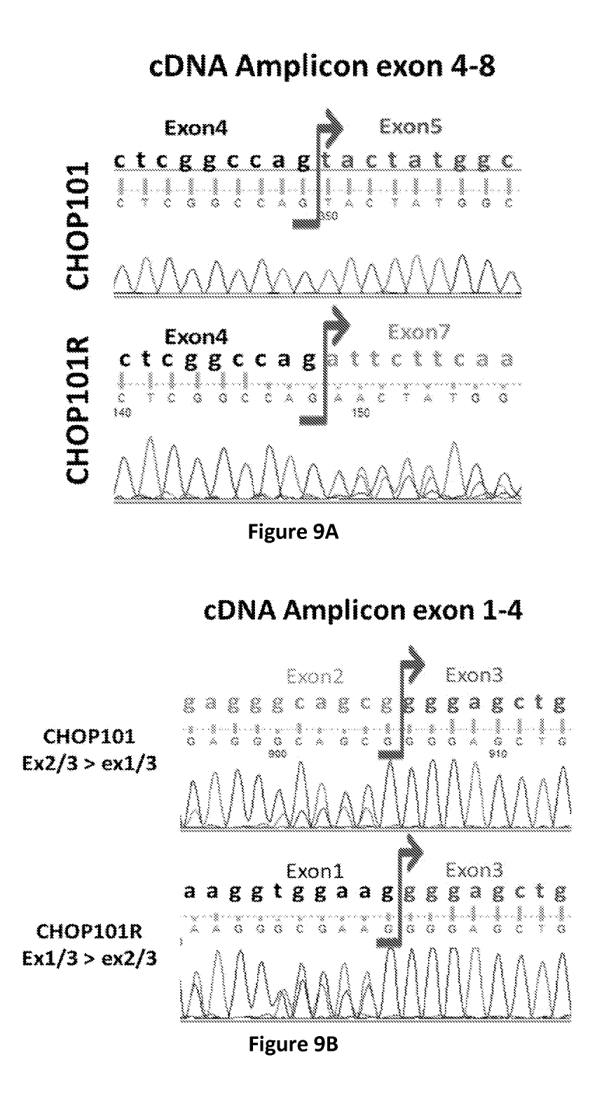

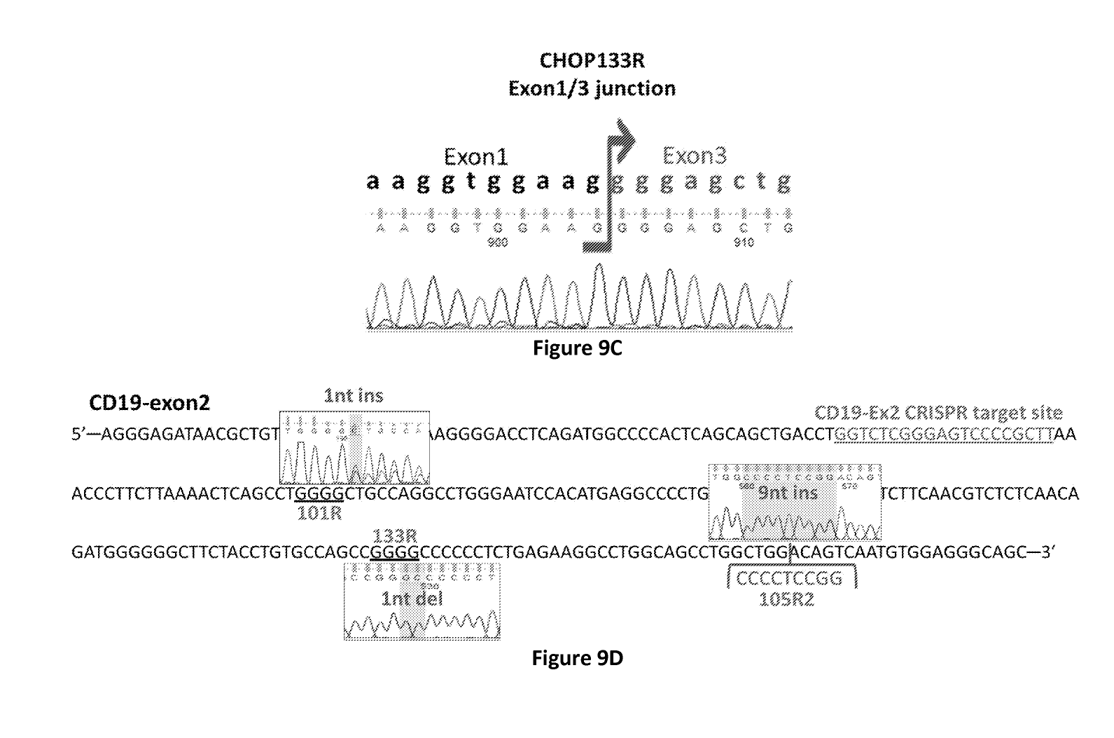

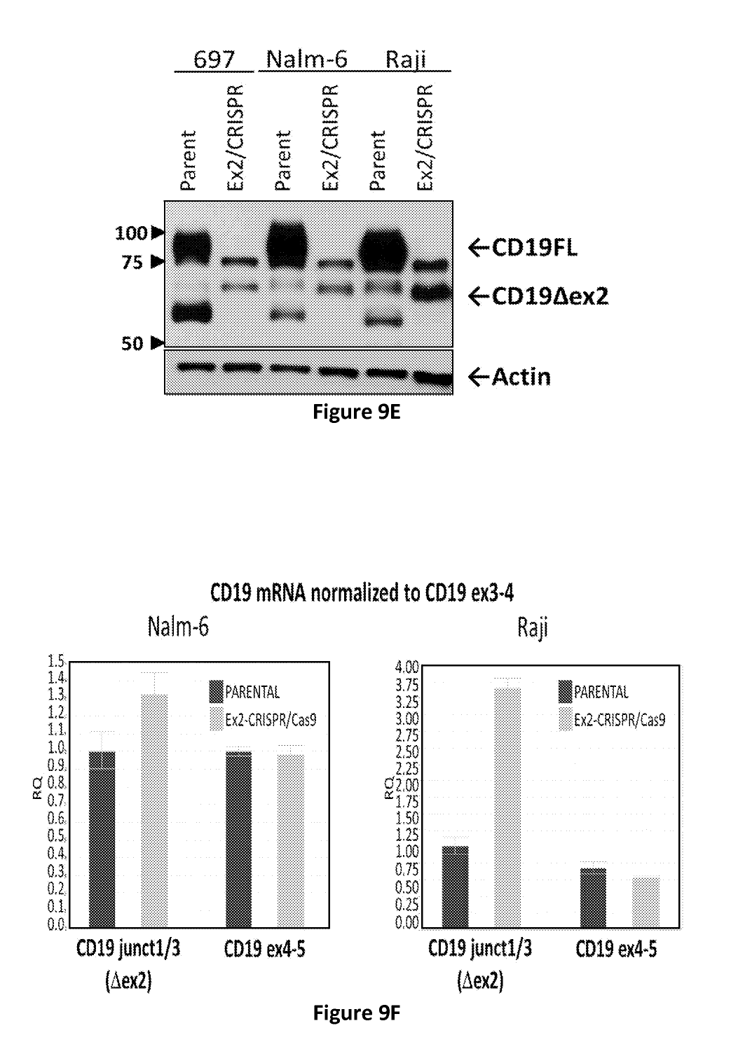

To further validate these changes, additional analyses on CHOP101/101R and CHOP105R1/105R2 were performed. The appearance of the Δex5-6 splicing variant in the relapsed samples was detected using very stringent radioactive low-cycle semi-quantitative RT-PCR (FIG. 8C) and confirmed by Sanger sequencing of RT-PCR products (FIG. 9A). When exon 1-4-specific primers were used, in both samples with CD19 epitope loss, there was 2.5-4.5-fold increased abundance of Δex2 and decreased levels of the full-length isoform (FIGS. 8D and 9B). The Δex2 isoform was also detectable in two other post-CART-19 leukemias for which no matching pre-treatment samples were available: CHOP107Ra and CHOP133R (FIGS. 8D and 9C).

To perform even more stringent quantification, a forward primer spanning the exon1-exon3 junction and thus specific for the Δex2 isoform was designed. By qRT-PCR, a sharp increase in exon 2 skipping in CHOP101R leukemia relative to CHOP101 was confirmed (FIG. 8E). To determine if some ex2 mRNA species retain exons 5-6, another pair of primers was designed to amplify the exon1-exon5 fragment. Using CHOP101R cDNA as template, fragments corresponding to both full-length and γex2 isoforms were observed (FIG. 8G). Sanger sequencing of these bands confirmed their makeup and revealed a frame-shift mutation present in the full-length (but not Δex2) isoform (FIG. 8G).

Consistent skipping of exon 2 prompted the re-evaluation of the seemingly deleterious frameshift mutations in exon 2 found in CHOP101R and CHOP133R (FIG. 9D). The CRISPR/Cas9 system with a guide RNA homologous to exon 2 was used to introduce double-stranded breaks in this exon in various B-cell lines and allowed them to repair by non-homologous end-joining. Frameshift events were selected for using sorting for CD19-negative cells and confirmed by sequencing. In all three cell lines tested [697, Nalm-6 (both B-ALL), and Raji (Burkitt's lymphoma)], frameshifts resulted in expression of a large CD19 protein isoform consistent in size with exon 2 skipping (FIG. 9E)—despite alternative stop codons downstream of the mutations site. Exon skipping was confirmed at the mRNA levels by qRT-PCR with exon1-exon 3 junction-specific primers (FIG. 8H and 9F). Thus, alternative splicing of exon 2 can override normally deleterious mutations.

The SRSF3 Splicing Factor Binds to and Regulates Inclusion of CD19 exon2

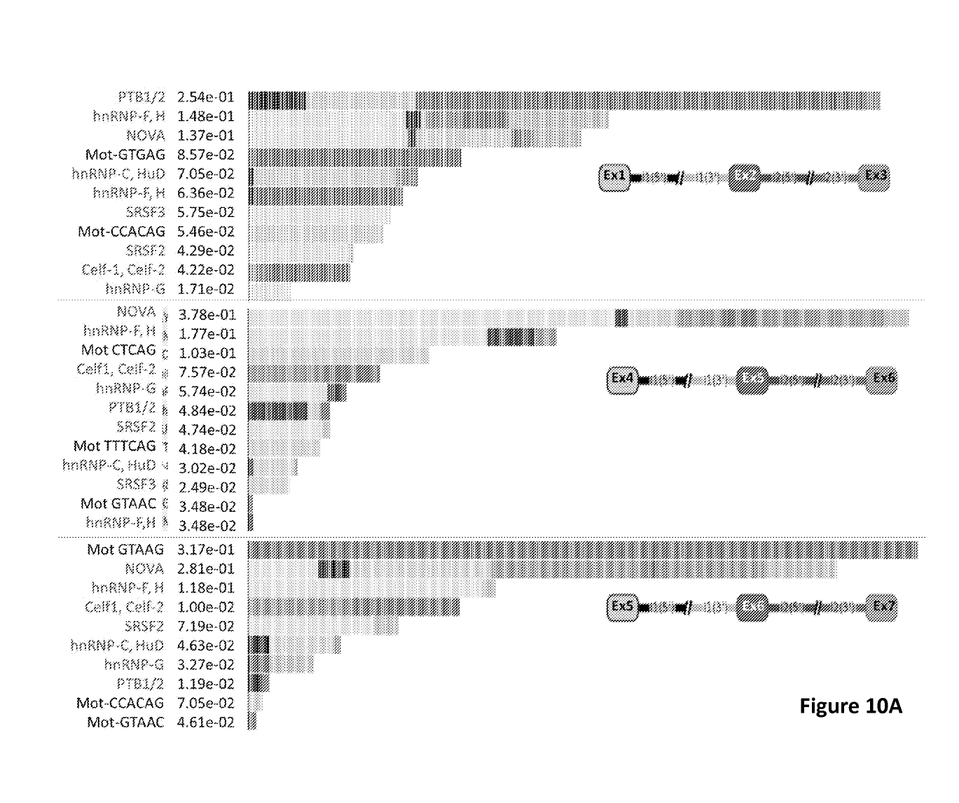

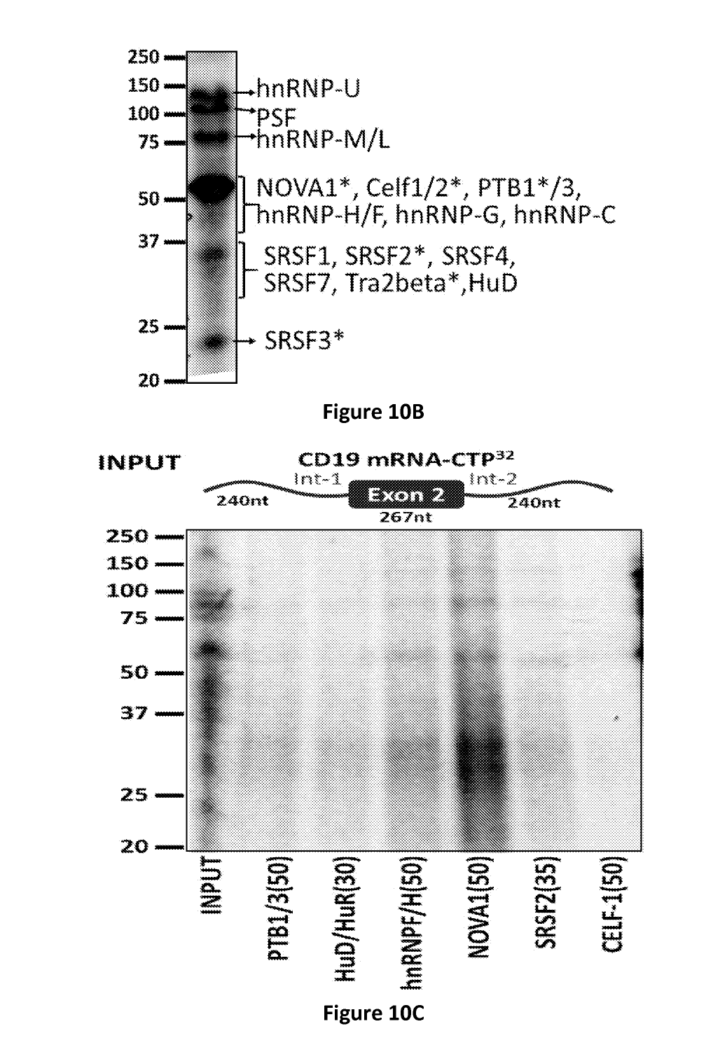

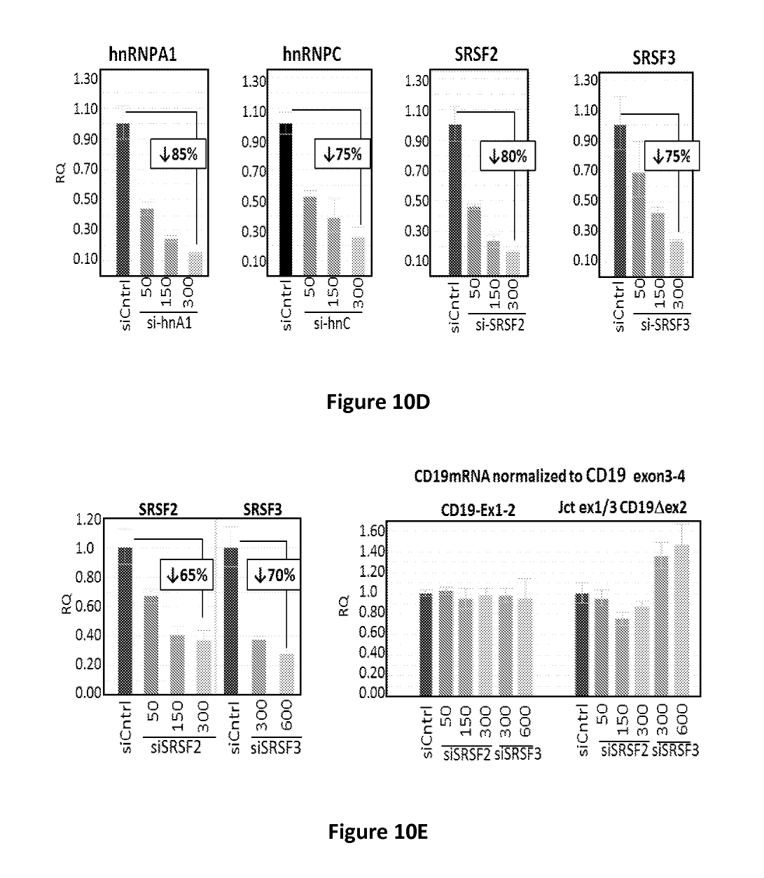

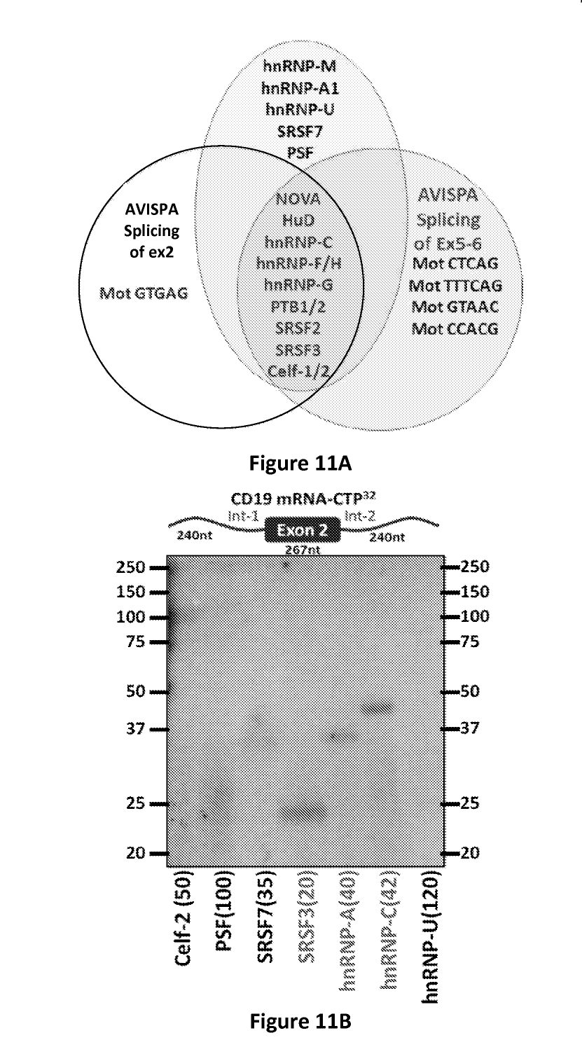

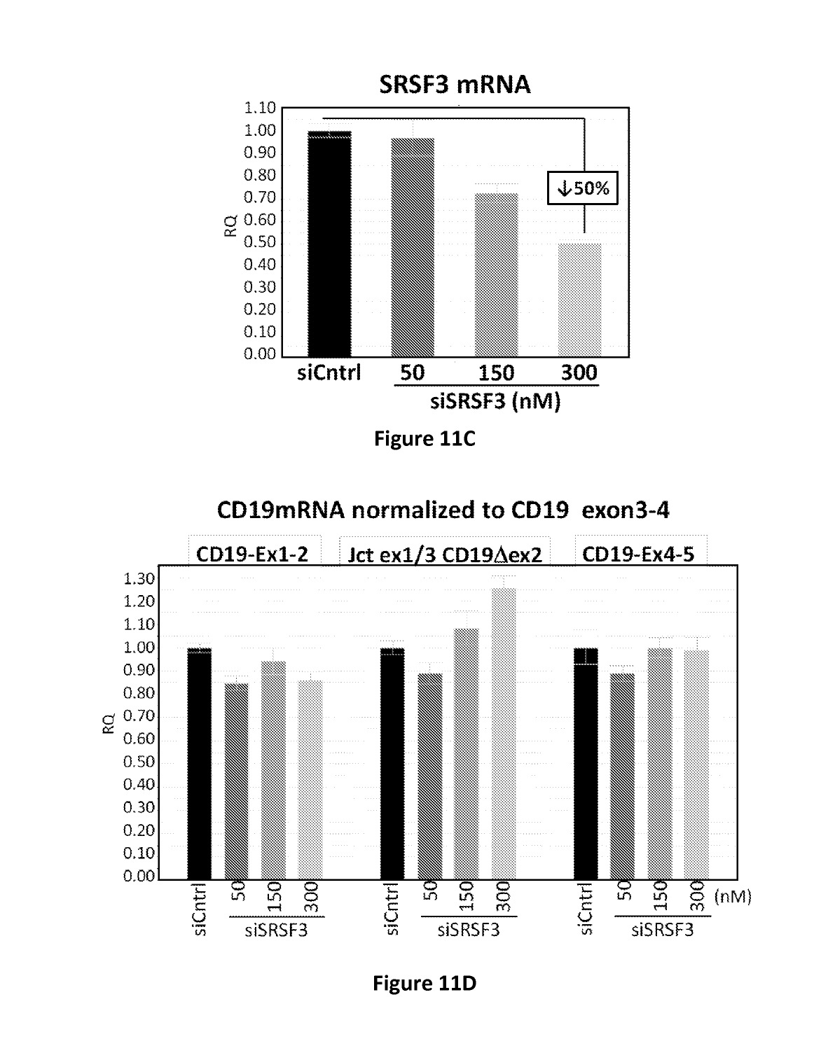

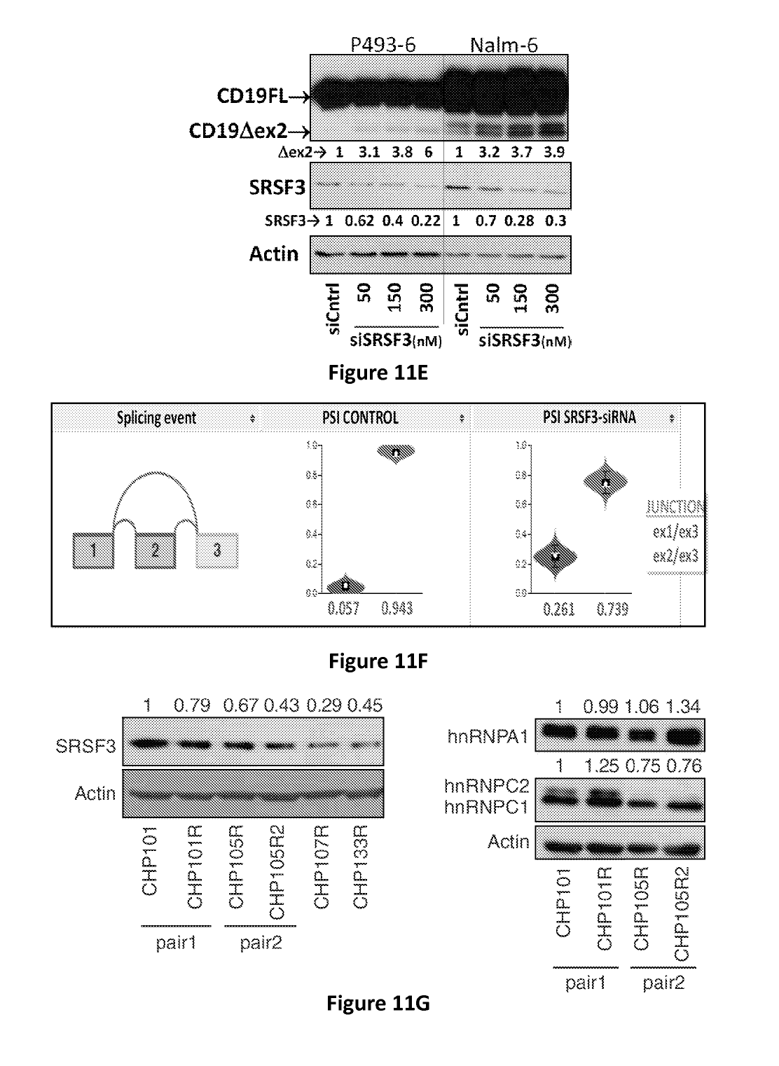

To understand the mechanism behind CD19 splicing, the AVISPA algorithm was used, which predicts RNA-binding proteins specific to particular intron-exon cassettes (Barash et al. (2013) Genome Biol., 14:R114). The predictions for exon 2 and 5-6 had a considerable overlap, consisting of NOVA, HuD, hnRNP-C, hnRNP-F/H, hnRNP-G, PTBP1/2, SRSF2, SRSF3, and Celf-1/2 (FIGS. 10A and 11A). T3-transcribed ³²P-labeled CD19 RNA containing introns 1/exon 2/intron 2 were generated and then cross-linked to protein lysates from B-ALL cells, and the labeled protein was separated by PAGE. The sizes of the observed bands were consistent with molecular weights of AVISPA-predicted as well as additional splice factors (SF) such as hnRNP-M, hnRNP-A1, hnRNP-U, SRSF7, and PSF (FIG. 10B). Nevertheless, most of these factors were negative by immunoprecipitation with SF-specific antibodies (FIG. 10C). In contrast, SRSF3 and hnRNP-A and -C were all positively confirmed in the same assay (FIG. 11B). To this list, SRSF2, which is thought to act in concert with SRSF3, has been added (Anko et al. (2012) Genome Biol., 13:R17).

To determine if any of these 3 SFs was involved in exon 2 alternative splicing, 3 siRNA pools were tested in B-lymphoid P493-6 cells amenable to efficient transfection (Psathas et al. (2013) Blood 122:4220-9) and efficient knockdown at the mRNA levels of SRSF3 and hnRNP-C were observed (FIG. 10D). However, only SRSF3 knockdown affected skipping of exon 2, as evidenced by the qRT-PCR assay (FIG. 11C). The knockdown experiment was repeated in Nalm-6 B-ALL cells where 65% to 75% decrease in SRSF2 and SRSF3 mRNA levels was achieved by siRNA transfection (FIG. 10E, left). Once again, only SRSF3 but not SRSF2 knockdown affected exon 2 processing (FIG. 10E, right). Most importantly, knockdown of SRSF3 resulted in increased abundance of the Δex2 protein isoform in both P493-6 and Nalm-6 B-ALL cells, as measured by immunoblotting for CD19 (FIG. 11D). To further confirm the role of SRSF3 in CD19 exon 2 retention, the publicly available GSE52834 dataset was mined where 22 RNA-binding proteins were knocked down in the GM19238 lymphoblastoid cell line. Of note, only knockdown of SRSF3 resulted in significant increase in CD19 exon 2 skipping (FIG. 11E). It was then asked whether any SRSF3 sites are present in exon 2 of CD19. The commonly used ESE-Finder tool does not include binding motifs for human SRSF3, because the consensus is not well defined. However, the Drosophila homolog of SRSF3, Rbp-1, is known to bind to the [A/T]CAAC[A/G] hexamer (Ray et al. (2013) Nature 499:172-7). Of note, this motif is found twice in CD19 exon 2, where it is not directly affected by de novo CD19 mutations (FIG. 10D).

To determine how SRSF3 function could be impaired in relapse leukemias, we assessed SRSF3 expression levels of in CHOP101/101R and CHOP105R1/105R2 matched sets. In both cases, relapsed leukemias expressed lower amounts of SRSF3. Also, two other post-CART-19 relapses CHOP107R and CHOP133R (for which matched baseline samples were not available) expressed even lower levels of this protein (FIG. 11F, top). In parallel, protein levels of hnRNPC1/C2 and hnRNPA1 were measured, but there was no consistent pattern of change for either of these splicing factors in paired post- versus pre-CART-19 samples (FIG. 11F, bottom). Taken together, these results indicate that SRSF3 insufficiency in relapsed leukemias could be at least partly responsible for the abundance of the CD19 Δex2 isoform.

The CD19 Δex2 Isoform Partially Rescues Defects Associated with CD19 Loss

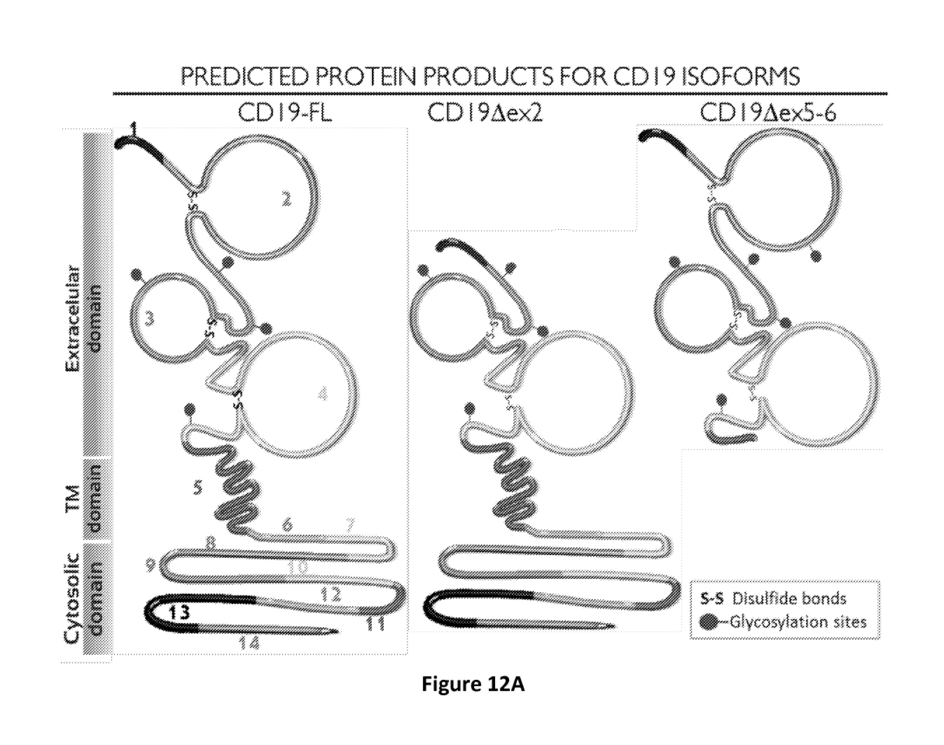

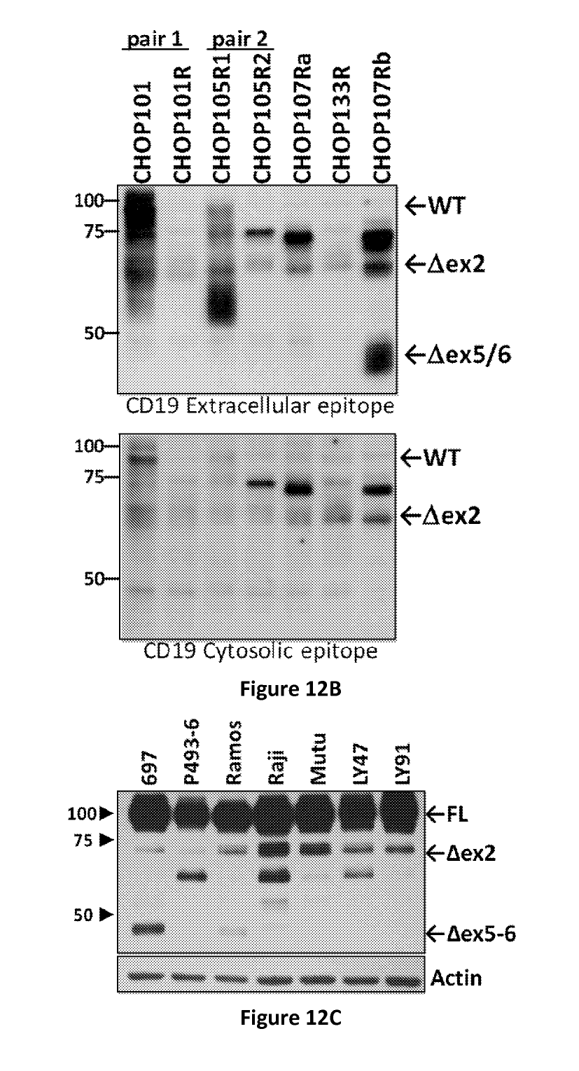

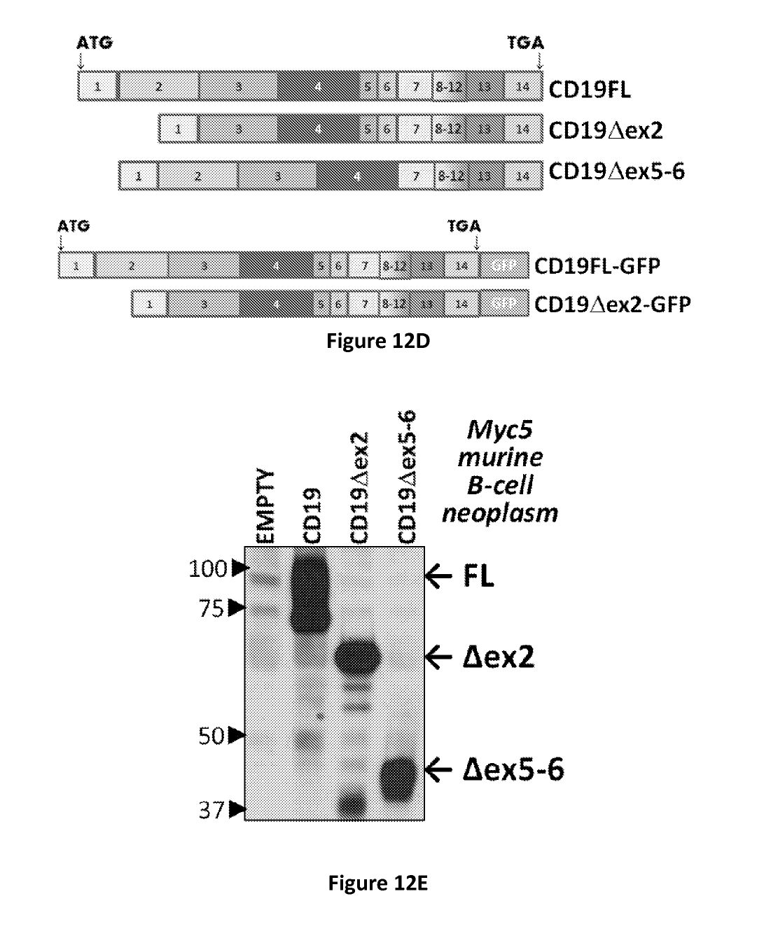

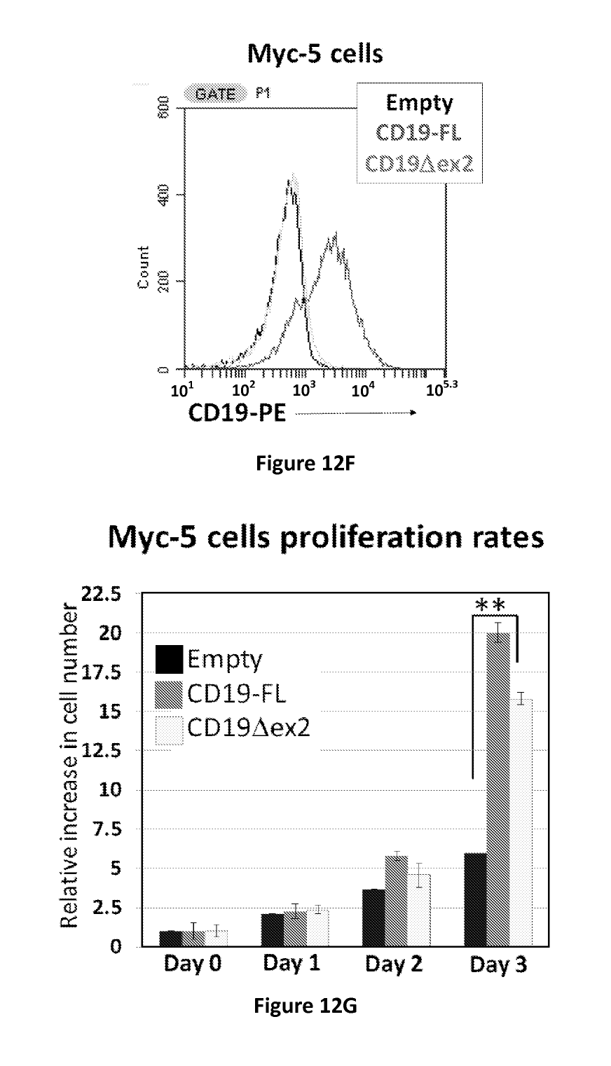

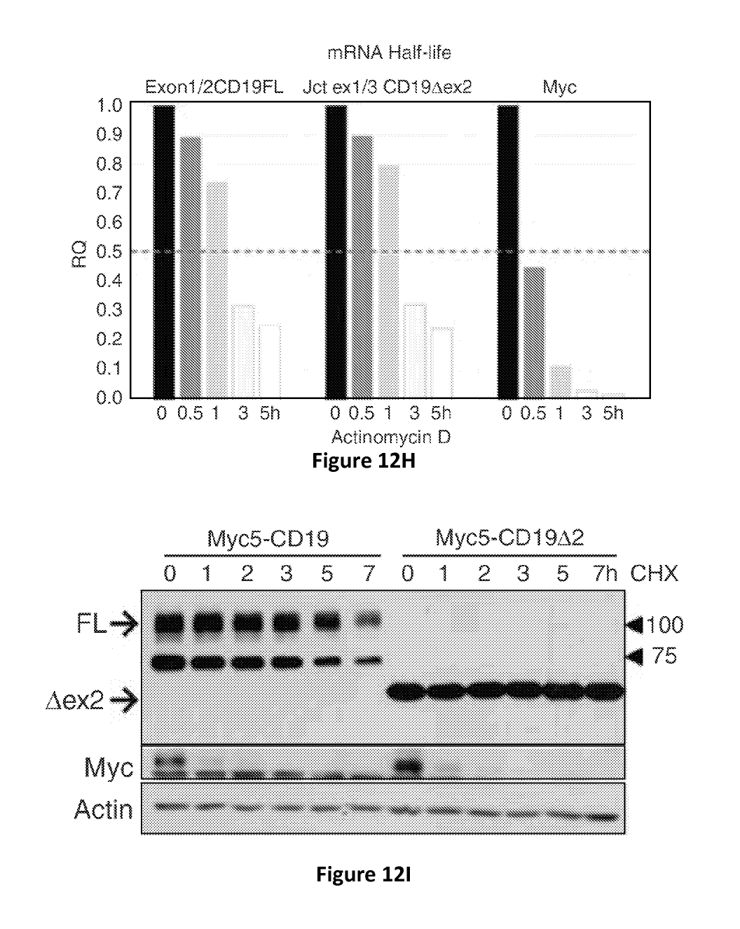

The detected alterations in exon inclusion should result in truncated CD19 variants, with profound implications for both CD19 functionality and CART-19 recognition. Skipping of exon 2 could compromise the FMC63 epitope targeted by the CAR (Nicholson et al. (1997) Mol. Immunol., 34:1157-65; Zola et al. (1991) Immunol. Cell Biol., 69(Pt 6):411-22) making it invisible to this immunotherapy. Skipping of exons 5 and 6 would result in premature termination, elimination of the transmembrane and the cytosolic domains (FIG. 12A). The expected truncated variants were readily detectable in leukemia cell lysates by immunoblotting using antibodies recognizing either extracellular or cytoplasmic epitopes (FIG. 12B), the hallmark of relapsed leukemias being the lack of the full-length isoform. Δex2 CD19 was also detectable in all human B-cell lines tested (FIG. 12C), attesting to its possible significance.

A series of CD19-encoding retroviruses (FIG. 12D, 12H) were generated and were transduced into the murine B-cell line Myc5, which had lost endogenous CD19 expression following silencing of its transcriptional regulator Pax5 (Yu et al. (2003) Blood 101:1950-5; Cozma et al. (2007) J. Clin. Invest., 117:2602-10). In this system, retrovirally encoded Δex2 and Δex5-6 isoforms were robustly expressed (FIG. 12E). Interestingly, when half-lives of CD19 protein isoforms were measured using treatment with cycloheximide, an increase in Δex2 protein stability was observed compared with the full-length isoform (FIG. 12I). As predicted, the Δex2 isoform was not recognized by the CD19 flow antibody (FIG. 12F). Importantly, in Myc5 cells restoration of full-length CD19 resulted in enhanced proliferation, and the Δex2 isoform (but not Δex5-6) partly recapitulated this growth phenotype (FIG. 12G).

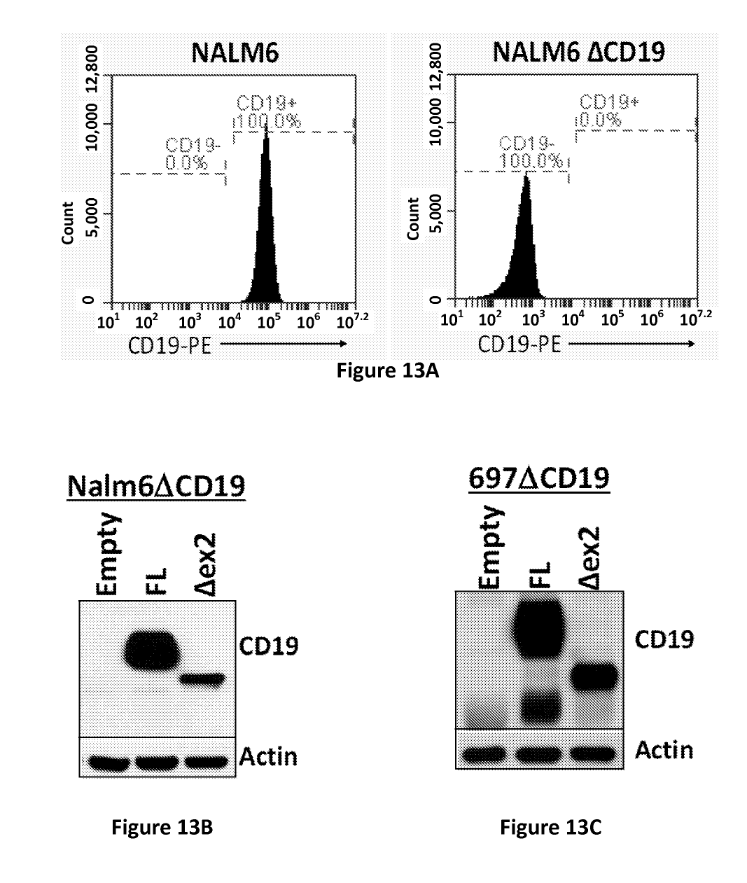

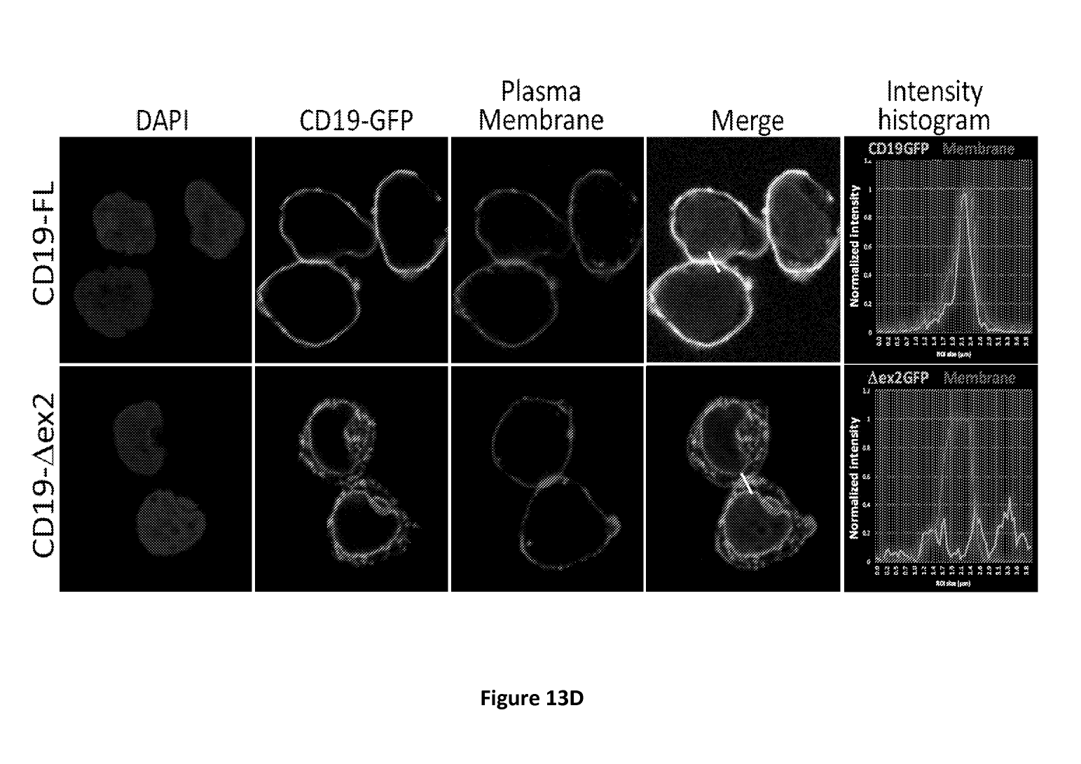

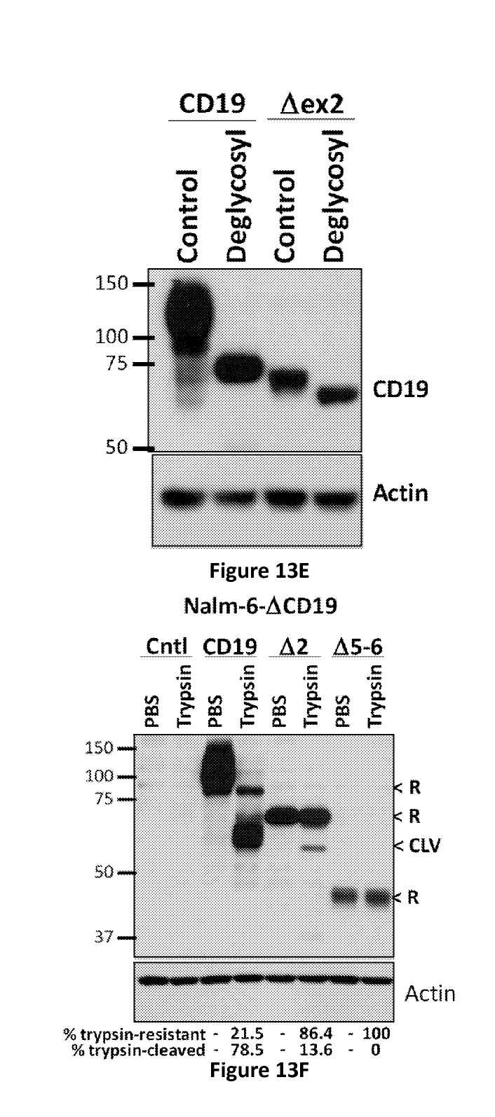

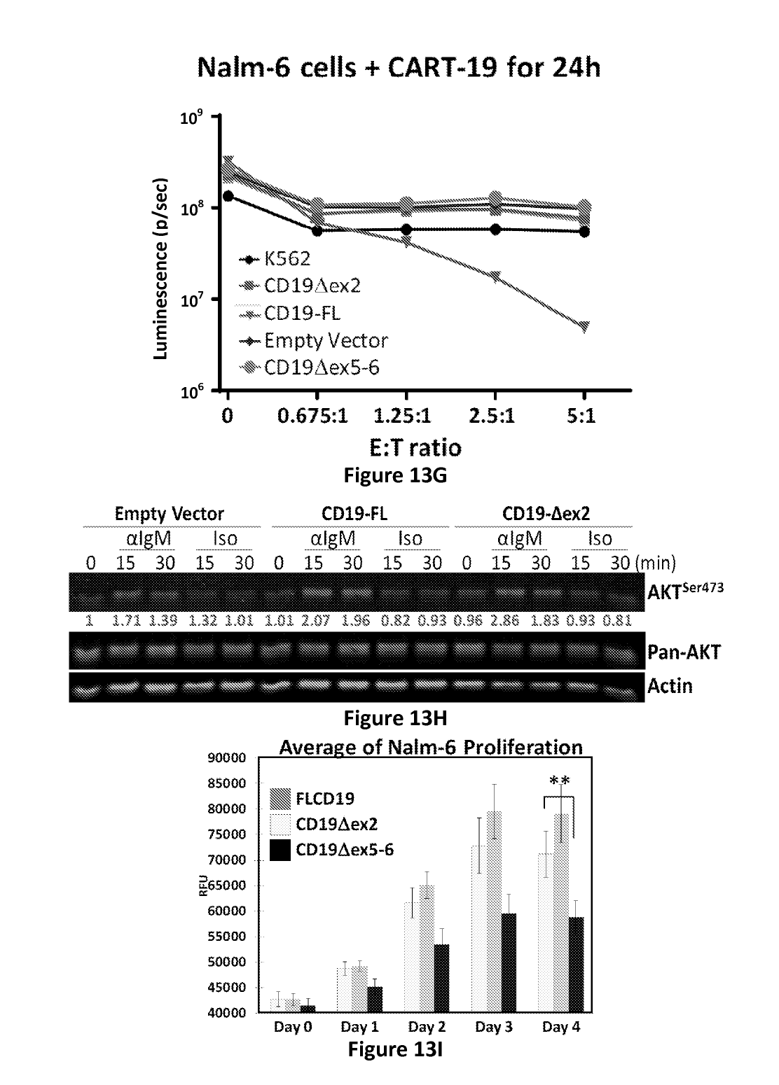

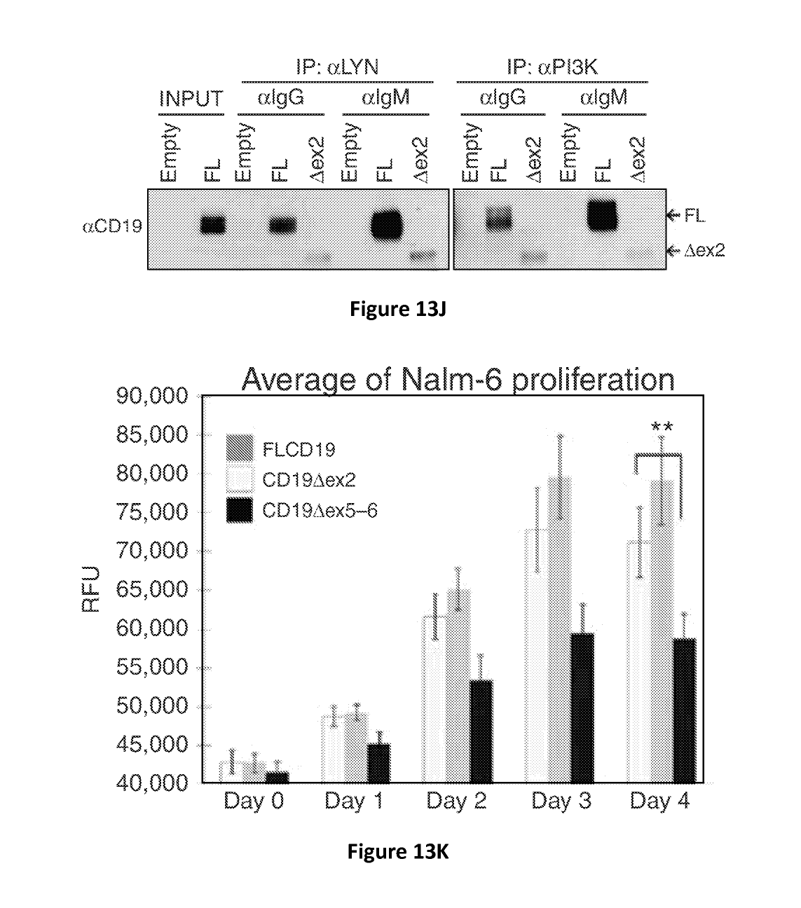

To establish relevance of this finding to human disease, Nalm6 and 697 B-ALL subclones were generated, in which the endogenous CD19 gene was knocked out using the CRISPR/Cas9 system, resulting in the loss of CD19 expression (FIG. 13A). The cells were then reconstituted with either full-length or Δex2 CD19 isoforms and confirmed robust protein expression by immunoblotting (FIG. 13B, 13C). The cells were also transduced with CD19-GFP fusion-encoding retroviruses (FIG. 12D). Unexpectedly, confocal microscopy revealed that unlike full-length CD19-GFP, which localizes exclusively to plasma membrane, the CD19 Δex2-GFP isoform is largely cytosolic. However, up to 10% of can still be found on the membrane (FIG. 13D). Further experiments were performed to validate the relevance of this fraction.

Glycosylation of CD19 is prerequisite for plasma membrane localization (van Zelm et al. (2010) J. Clin. Invest., 120:1265-74), and unlike its intracellular precursor, plasma membrane-bound CD19 is susceptible to extracellular cleavage by trypsin (Shoham et al. (2006) Mol. Cell Biol., 26:1373-85). To determine whether the Δex2 isoform is glycosylated, whole cell protein lysates obtained from CD19 retrovirus-transduced cultures were treated with a mix of glycosylases followed by Western blotting. Just like its full-length counterpart, the Δex2 isoform was reduced in size upon treatment (FIG. 13E) indicating that it is glycosylated and that some of it could be transported to the plasma membrane. To quantitate the membrane-bound fraction, reconstituted live cells were incubated with trypsin. As expected, almost all of the full-length CD19 was cleaved by trypsin while most of the Δex2 isoform and all of the Δex5-6 isoform retained its original size. However, over 10% of the CD19 Δex2 protein was sensitive to trypsinization, fully consistent with the results of confocal microscopy (FIG. 13F). This in principle could be sufficient to trigger killing by CART-19 cells. However, when exposed to CART-19, only the full-length CD19 cultures were killed, while CD19 Δex2 transduced cells remained fully viable (FIG. 13G), confirming the loss of the cognate CART-19 epitope.

To test whether this plasma membrane-associated fraction is functional, it was determined whether it contributes to tonic or antigen-driven pre-BCR signaling by directly recruiting PI3 and Src family tyrosine kinases, such as Lyn (Psathas et al. (2013) Blood 122:4220-9; Depoil et al. (2008) Nat. Immunol., 9:63-72; Buchner et al. (2014) Curr. Opin. Hematol., 21:341-9). In both full length- and Δex2-reconstituted cells, PI3K and Lyn coimmunoprecipitated with CD19, albeit less abundantly in the latter case, reflecting a much smaller pool of plasma membrane-associated Δex2. When pre-BCR was ligated with the anti-IgM antibody, there was an increase in Δex2 CD19-Lyn binding, although the amount of Δex2 CD19-bound PI3K was reduced (FIG. 13J). Moreover, similarly to reconstituted murine Myc5 cells, human Δex2 cells grew in culture almost as rapidly as their full-length CD19 counterparts and significantly faster than control CD19 Δex5-6 cells (FIG. 13K). In principle, the presence of functional Δex2 on the plasma membrane could be sufficient to trigger killing by CART-19 cells. However, when exposed to CART-19, only the full-length CD19 cultures were killed, whereas CD19 Δex2-transduced cells remained fully viable (FIG. 13I), confirming the loss of the cognate CART-19 epitope.

The above data addresses the important clinical issue of resistance to CART-19 and establishes a novel combinatory mechanism by which its cognate epitope could be removed from the cell surface without discarding the target protein entirely. This mechanism is based on the clustering of nonsense and missense mutations in exon 2 of CD19. Distributed frameshift mutations would have prevented CD19 protein expression but also left the leukemic cells without the important activator of PI3K and SFTK signaling. In contrast, frameshift mutations clustered in the non-constitutive exon 2 eliminate full-length CD19 but allow expression of the Δex2 isoform. This isoform does not trigger killing by CART-19, at least not at physiological levels. At the same time, it was found to be even more stable than full-length CD19, which could be due to either the presence of a degron within exon 2-encoded amino acid sequence or mislocalization of Δex2 CD19 protein away from its normal degradation pathways. Moreover, this isoform at least partly rescues defects in cell proliferation and pre-BCR signaling associated with CD19 loss. Thus, its retention in relapsed B-ALL is highly advantageous for leukemic cells, whether or not they carry de novo mutations in exon 2.

It is well-known that splicing occurs co-transcriptionally when pre-mRNA is still in the vicinity of chromatin, which can influence intron removal (Hnilicova et al. (2011) Nucleus 2:182-8; Iannone et al. (2013) Chromosoma 122:465-74; Naftelberg et al. (2015) Ann. Rev. Biochem., 84:165-98). Certain histone modifications are enriched on chromatin associated with exonic sequences (Brown et al. (2012) Hum. Mol. Genet., 21:R90-R6) and spliceosome machinery is recruited via cofactors recognizing histone modifications and/or associates directly with modified histones. For example, H3K36me3 interacts with PSIP1, which then recruits various splice factors, including SRSF3 (Pradeepa et al. (2012) PLoS Genet., 8:e1002717). The underlying mechanism notwithstanding, it is becoming clear that alterations in splicing factors are important drivers of hematological malignancies, as evidenced by the discovery of acquired mutations in the splicing factor SF3B1 gene in chronic lymphocytic leukemia (Quesada et al. (2012) Nat. Genet., 44:47-52) and myelodysplasia (Yoshida et al. (2011) Nature 478:64-9).

Whether hematological malignancies are driven by global deregulation of splicing or by alterations in select target genes is not known. The instant data underscore the importance of splicing alterations at the level of individual genes, at least in the context of disease progression. Similarly, in the realm of solid tumors, BRAF(V600E) splicing variants lacking the RAS-binding domain were found in ⅓ of melanomas with acquired resistance to vemurafenib (Poulikakos et al. (2011) Nature 480:387-90). The existence of such splicing-based adaptive mechanisms suggests that future CARs and other antibody-based therapeutics should be designed to target essential exons, as a way to prevent immunological escape (Mittal et al. (2014) Curr. Opin. Immunol., 27:16-25).

On the other hand, it is conceivable that splicing is globally deregulated in B-ALL, either owing to downregulation of SRSF3 and related splicing factors or due to pervasive epigenetic changes. In that case, it should be possible to define a set of genes that are alternatively spliced in B-ALL vs. normal B-cells and encode extracellular epitopes. Such epitopes could be targets for completely new chimeric antigen receptors capable of killing B-ALL blasts while sparing normal B-cells, with the selectivity CART-19 does not possess.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of inhibiting a B-cell neoplasm in a subject in need thereof, wherein said B-cell neoplasm expresses a CD19 isoform, said method comprising administering to the subject a therapeutically effective amount of at least one Src family kinase (SFK) inhibitor and/or at least one chimeric antigen receptor-modified T cell which recognizes the ectodomain of said CD19 isoform, CD20, or CD22.

2. The method of claim 1, wherein said B-cell neoplasm is a lymphoma or B-cell acute lymphoblastic leukemia.

3. The method of claim 1, wherein said B-cell neoplasm is a relapse after CART19 therapy.

4. The method of claim 1, comprising administering to the subject a therapeutically effective amount of a Src family kinase (SFK) inhibitor.

5. The method of claim 4, wherein said SFK inhibitor is a Lyn inhibitor.

6. The method of claim 4, wherein said SFK inhibitor is dasatinib.

7. The method of claim 1, further comprising the administration of at least one other chemotherapeutic agent or radiation therapy to the subject.

8. The method of claim 1, comprising administering to the subject at least one chimeric antigen receptor-modified T cell which recognizes the ectodomain of said CD19 isoform, CD20, or CD22.

9. The method of claim 8, comprising administering to the subject a chimeric antigen receptor-modified T cell which recognizes the ectodomain of said CD19 isoform.

10. The method of claim 1, wherein said CD19 isoform comprises a deletion of exon 2, 5, and/or 6.

11. The method of claim 1, wherein said B cell neoplasm does not substantially express wild-type CD19.

12. The method of claim 9, wherein said CD19 isoform comprises a deletion of exon 2, 5, and/or 6.

13. The method of claim 12, wherein said CD19 isoform comprises a deletion of exon 2.

14. The methods of claim 1, wherein said method further comprises determining the isoform of CD19 expressed by the B-cell neoplasm prior to treatment of the subject.


SEQUENCE LISTING (80 sequences)

SEQ ID NO: 1 - DNA, 244 bp, Artificial Sequence
  DNA Fragment EcoRI/BglII CD19-ex2
gaattcaccaccatgccacctcctcgcctcctcttcttcctcctcttcctcacccccatggaagtcaggcccgaggaacctctagtggtgaaggtggaagaggagctgttccggtggaatgtttcggacctaggtggcctgggctgtggcctgaagaacaggtcctcagagggccccagctccccttccgggaagctcatgagccccaagctgtatgtgtgggccaaagaccgccctgagatct

SEQ ID NO: 2 - DNA, 1049 bp, Artificial Sequence
  DNA Fragment BglII/XhoI CD19-ex5-6
agatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcgag

SEQ ID NO: 3 - DNA, 20 bp, Artificial Sequence
  Primer
gcaggggaatgacatgctct

SEQ ID NO: 4 - DNA, 20 bp, Artificial Sequence
  Primer
attagcccagtgtccagcac

SEQ ID NO: 5 - DNA, 20 bp, Artificial Sequence
  Primer
atgatgtgctggacactggg

SEQ ID NO: 6 - DNA, 20 bp, Artificial Sequence
  Primer
tccctgccacgctgttttat

SEQ ID NO: 7 - DNA, 20 bp, Artificial Sequence
  Primer
tctactccaaggggctcaca

SEQ ID NO: 8 - DNA, 20 bp, Artificial Sequence
  Primer
actgcagcacagcgttatct

SEQ ID NO: 9 - DNA, 20 bp, Artificial Sequence
  Primer
ggagagtctgaccaccatgc

SEQ ID NO: 10 - DNA, 20 bp, Artificial Sequence
  Primer
ggacacagagtcagggggta

SEQ ID NO: 11 - DNA, 20 bp, Artificial Sequence
  Primer
gagccccaagctgtatgtgt

SEQ ID NO: 12 - DNA, 20 bp, Artificial Sequence
  Primer
gaagttggggtttggggtga

SEQ ID NO: 13 - DNA, 20 bp, Artificial Sequence
  Primer
ttccctcgcttccaagacac

SEQ ID NO: 14 - DNA, 20 bp, Artificial Sequence
  Primer
gggattgtcacagaccctgg

SEQ ID NO: 15 - DNA, 20 bp, Artificial Sequence
  Primer
tgccagggtctgtgacaatc

SEQ ID NO: 16 - DNA, 20 bp, Artificial Sequence
  Primer
aggctagaggaagactgggg

SEQ ID NO: 17 - DNA, 20 bp, Artificial Sequence
  Primer
ccccagtcttcctctagcct

SEQ ID NO: 18 - DNA, 20 bp, Artificial Sequence
  Primer
gaagaggaagtgcacggtga

SEQ ID NO: 19 - DNA, 20 bp, Artificial Sequence
  Primer
aatgactgaccccaccagga

SEQ ID NO: 20 - DNA, 20 bp, Artificial Sequence
  Primer
tgctcgggtttccataagac

SEQ ID NO: 21 - DNA, 20 bp, Artificial Sequence
  Primer
gaacttcgccccagaactga

SEQ ID NO: 22 - DNA, 20 bp, Artificial Sequence
  Primer
aagctgcagagtaagctggg

SEQ ID NO: 23 - DNA, 17 bp, Artificial Sequence
  Primer
cagccgggaagcaacct

SEQ ID NO: 24 - DNA, 20 bp, Artificial Sequence
  Primer
attgctccagaggttggcat

SEQ ID NO: 25 - DNA, 25 bp, Artificial Sequence
  Primer
aatttttgtttttaaaggatttttt

SEQ ID NO: 26 - DNA, 25 bp, Artificial Sequence
  Primer
ctacaaataacaaaacctccacttc

SEQ ID NO: 27 - DNA, 27 bp, Artificial Sequence
  Primer
ttgggtttttttaaaataatttttttt

SEQ ID NO: 28 - DNA, 26 bp, Artificial Sequence
  Primer
aacaaaaaccaaacacaataatacac

SEQ ID NO: 29 - DNA, 25 bp, Artificial Sequence
  Primer
agttttattttggtgtttaggttgg

SEQ ID NO: 30 - DNA, 25 bp, Artificial Sequence
  Primer
aaacacataaactccttctcaaaaa

SEQ ID NO: 31 - DNA, 30 bp, Artificial Sequence
  Primer
tttgtgtagaaaatagaaatgaataaataa

SEQ ID NO: 32 - DNA, 25 bp, Artificial Sequence
  Primer
aacaaaaacctaaaaaacactcaac

SEQ ID NO: 33 - DNA, 30 bp, Artificial Sequence
  Primer
gtagatatttatggttgagtgttttttagg

SEQ ID NO: 34 - DNA, 30 bp, Artificial Sequence
  Primer
ttcctaaattttacaaataaaaaaataaaa

SEQ ID NO: 35 - DNA, 25 bp, Artificial Sequence
  Primer
ggtttttattagattttggggtttt

SEQ ID NO: 36 - DNA, 25 bp, Artificial Sequence
  Primer
taatatctctacaaccttccccaac

SEQ ID NO: 37 - DNA, 24 bp, Artificial Sequence
  Primer
ccgaggaacctctagtggtgaagg

SEQ ID NO: 38 - DNA, 23 bp, Artificial Sequence
  Primer
ccacaggacagccagagtgtgga

SEQ ID NO: 39 - DNA, 25 bp, Artificial Sequence
  Primer
cctaagtcattgctgagcctagagc

SEQ ID NO: 40 - DNA, 24 bp, Artificial Sequence
  Primer
cgctgctcgggtttccataagacg

SEQ ID NO: 41 - DNA, 749 bp, Artificial Sequence
  XhoI/NotI CD19-int1-ex2-int2
ctcgaggaagggattgaggctggaaacttgagttgtggctgggtgtccttggctgagtaacttaccctctctgagcctccattttcttatttgtaaaattcaggaaagggttggaaggactctgccggctcctccactcccagcttttggagtcctctgctctataacctggtgtgaggagtcggggggcttggaggtcccccccacccatgcccacacctctctccctctctctccacagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggtgagggccgggctggggcaggggcaggaggagagaagggaggccaccatggacagaagaggtccgcggccacaatggagctggagagaggggctggagggattgagggcgaaactcggagctaggtgggcagactcctggggcttcgtggcttcagtatgagctgcttcctgtccctctacctctcactgtcttctctctctctgcgggtctttgtctctatttatctctgtgcggccgc

SEQ ID NO: 42 - DNA, 20 bp, Artificial Sequence
  Primer
ggagagtctgaccaccatgc

SEQ ID NO: 43 - DNA, 20 bp, Artificial Sequence
  Primer
ggacacagagtcagggggta

SEQ ID NO: 44 - DNA, 20 bp, Artificial Sequence
  Primer
aaggggcctaagtcattgct

SEQ ID NO: 45 - DNA, 20 bp, Artificial Sequence
  Primer
tgctcgggtttccataagac

SEQ ID NO: 46 - DNA, 17 bp, Artificial Sequence
  Primer
ggcccgaggaacctcta

SEQ ID NO: 47 - DNA, 20 bp, Artificial Sequence
  Primer
cagcagccagtgccatagta

SEQ ID NO: 48 - DNA, 20 bp, Artificial Sequence
  Primer
cacccggctttgcttttgtt

SEQ ID NO: 49 - DNA, 20 bp, Artificial Sequence
  Primer
cggcagccacatagtgttct

SEQ ID NO: 50 - DNA, 16 bp, Artificial Sequence
  Primer
cggagccgcagcccta

SEQ ID NO: 51 - DNA, 20 bp, Artificial Sequence
  Primer
ggtcgaccgagatcgagaac

SEQ ID NO: 52 - DNA, 20 bp, Artificial Sequence
  Primer
agaacccgggagtaggagac

SEQ ID NO: 53 - DNA, 21 bp, Artificial Sequence
  Primer
agccgaaaatgtagctgaaga

SEQ ID NO: 54 - DNA, 20 bp, Artificial Sequence
  Primer
ggtaggctggcagatacgtt

SEQ ID NO: 55 - DNA, 20 bp, Artificial Sequence
  Primer
taacgatgcttcttcggcgg

SEQ ID NO: 56 - DNA, 19 bp, Artificial Sequence
  Primer
agcatcccccaaagttcac

SEQ ID NO: 57 - DNA, 21 bp, Artificial Sequence
  Primer
aagggacttcctgtaacaacg

SEQ ID NO: 58 - DNA, 20 bp, Artificial Sequence
  Primer
ggagagtctgaccaccatgc

SEQ ID NO: 59 - DNA, 20 bp, Artificial Sequence
  Primer
actgcagcacagcgttatct

SEQ ID NO: 60 - DNA, 20 bp, Artificial Sequence
  Primer
gagccccaagctgtatgtgt

SEQ ID NO: 61 - DNA, 20 bp, Artificial Sequence
  Primer
ggacacagagtcagggggta

SEQ ID NO: 62 - DNA, 22 bp, Artificial Sequence
  Primer
gcctcctcttcttcctcctctt

SEQ ID NO: 63 - DNA, 25 bp, Artificial Sequence
  Primer
ccggaacagctccccttccaccttc

SEQ ID NO: 64 - DNA, 20 bp, Artificial Sequence
  Primer
aaggggcctaagtcattgct

SEQ ID NO: 65 - DNA, 20 bp, Artificial Sequence
  Primer
cagcagccagtgccatagta

SEQ ID NO: 66 - DNA, 20 bp, Artificial Sequence
  Primer
ccccaccaggagattcttca

SEQ ID NO: 67 - DNA, 20 bp, Artificial Sequence
  Primer
tgctcgggtttccataagac

SEQ ID NO: 68 - DNA, 34 bp, Artificial Sequence
  Synthetic Sequence
tagtggtgaaggtggaaggggagctgttccggtg

SEQ ID NO: 69 - DNA, 27 bp, Artificial Sequence
  Synthetic Sequence
actcagcctggggctgccagggctggg

SEQ ID NO: 70 - DNA, 18 bp, Artificial Sequence
  Synthetic Sequence
ctcggccagtactatggc

SEQ ID NO: 71 - DNA, 18 bp, Artificial Sequence
  Synthetic Sequence
ctcggccagattcttcaa

SEQ ID NO: 72 - DNA, 18 bp, Artificial Sequence
  Synthetic Sequence
gagggcagcggggagctg

SEQ ID NO: 73 - DNA, 18 bp, Artificial Sequence
  Synthetic Sequence
aaggtggaaggggagctg

SEQ ID NO: 74 - DNA, 266 bp, Artificial Sequence
  CD19-exon2
agggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagc

SEQ ID NO: 75 - DNA, 11 bp, Artificial Sequence
  Synthetic Sequence
tggggctgcca

SEQ ID NO: 76 - DNA, 17 bp, Artificial Sequence
  Synthetic Sequence
tggcccctccggacagt

SEQ ID NO: 77 - DNA, 12 bp, Artificial Sequence
  Synthetic Sequence
ccgggcccccct

SEQ ID NO: 78 - DNA, 9 bp, Artificial Sequence
  Synthetic Sequence
cccctccgg

SEQ ID NO: 79 - DNA, 1965 bp, Homo sapiens
aggcccctgcctgccccagcatcccctgcgcgaagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaatcctaaaaaaaaaaa

SEQ ID NO: 80 - protein, 556 aa, Homo Sapiens [sequence omitted]